SEQUENCE LISTING (15 sequences)

SEQ ID NO: 1 - DNA, 459 bp, synthetic construct
  misc_feature 1..459: note = pETDuet-1-based expression construct
  source 1..459: mol_type = other DNA
atgggcagcagccatcaccatcatcaccacagccaggatccgggtagctatgcgctgggtccgtatcagatctctgctccgcagctgccggcatataacggtcagactgttggtactttctattatgttaacgatgctggcggtttagaaagcaaagttttcagctctggtggtccgaccccgtatccgaactatgctaacgctggtcacgttgaaggtcagtctgctctgttcatgcgtgataacggtatctctgaaggtctggttttccataacaacccggaaggtacctgtggtttttgtgttaacatgaccgaaaccctgctgccggaaaacgctaaaatgaccgttgttccgccggaaggtgcgattccggttaaacgtggtgctaccggtgaaaccaaagttttcaccggtaactctaactctccgaaatctccgaccaaaggtggttgctaa

SEQ ID NO: 2 - protein, 152 aa, synthetic construct
  REGION 1..152: note = pETDuet-1-based expression construct
MGSSHHHHHHSQDPGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 3 - DNA, 372 bp, synthetic construct
  misc_feature 1..372: note = DddAI encoding sequence
  source 1..372: mol_type = other DNA
atgtatgcggatgactttgacggggaaattgagattgatgaagttgatagcctagttgagtttctgagccgtcgtccggcgttcgatgcgaacaacttcgttctgaccttcgaagaaagcggcttcccgcagctgaacatcttcgcgaaaaacgatatcgcggttgtttactacatggatatcggcgaaaacttcgttagcaaaggcaacagcgcgagcggcggcaccgaaaaattctacgaaaacaaactgggcggcgaagttgatctgagcaaagattgcgttgttagcaaagaacagatgatcgaagcggcgaaacagttcttcgcgaccaaacagcgtccggaacagctgacctggagcgaactgtaa

SEQ ID NO: 4 - protein, 123 aa, synthetic construct
  REGION 1..123: note = DddAI encoding sequence
MYADDFDGEIEIDEVDSLVEFLSRRPAFDANNFVLTFEESGFPQLNIFAKNDIAVVYYMDIGENFVSKGNSASGGTEKFYENKLGGEVDLSKDCVVSKEQMIEAAKQFFATKQRPEQLTWSEL

SEQ ID NO: 5 - DNA, 459 bp, synthetic construct
  misc_feature 1..459: note = pETDuet-1-based expression construct
  source 1..459: mol_type = other DNA
atgggcagcagccatcaccatcatcaccacagccaggatccgggtagctatgcgctgggtccgtatcagatctctgctccgcagctgccggcatataacggtcagactgttggtactttctattatgttaacgatgctggcggtttagaaagcaaagttttcatctctggtggtccgaccccgtatccgaactatgttagcgctggtcacgttgaaggtcagtctgctctgttcatgcgtgataacggtatctctgaaggtctggttttccataacaacccgaaaggtacctgtggtttttgtgttaacatgatcgaaaccctgctgccggaaaacgctaaaatgaccgttgttccgccggaaggtgcgattccggttaaacgtggtgctaccggtgaaaccaaagttttcatcggtaactctaactctccgaaatctccgaccaaaggtggttgctaa

SEQ ID NO: 6 - protein, 138 aa, synthetic construct
  REGION 1..138: note = pETDuet-1-based expression construct
GSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAKMTVVPPEGAIPVKRGATGETKVFIGNSNSPKSPTKGGC

SEQ ID NO: 7 - DNA, 372 bp, synthetic construct; identical to SEQ ID NO: 3
  misc_feature 1..372: note = DddAI encoding sequence
  source 1..372: mol_type = other DNA

SEQ ID NO: 8 - protein, 123 aa, synthetic construct; identical to SEQ ID NO: 4
  REGION 1..123: note = DddAI encoding sequence

SEQ ID NO: 9 - DNA, 408 bp, synthetic construct
  misc_feature 1..408: note = pETDuet-1-based expression construct
  source 1..408: mol_type = other DNA
atgggcagcagccatcaccatcatcaccacagccaggatcctggttggaaattttctaacggtaaacgccgtccgccgcacaaagcaacggtaactgtgaccgataaaaacggtgtcgttaaacacaaaagcaacctggtttctggcaacatgactgaagccgaaaagaaactgggcttcccgaacaactccctggcgacccacaccgaaaaccgtgctacccgcctgatcgatctgaaccaaggtgatactatgctgatcgagggccaataccgtccgtgtccacgttgtaaaggtgcaatgcgcgtgaaagcggaggaatccggtgcgaaagtgatctacacctggccagaagatggtgacctgaaaaaacgtgaatgggaaggcactccgtgcgacaaaaaataa

SEQ ID NO: 10 - protein, 135 aa, synthetic construct
  REGION 1..135: note = pETDuet-1-based expression construct
MGSSHHHHHHSQDPGWKFSNGKRRPPHKATVTVTDKNGVVKHKSNLVSGNMTEAEKKLGFPNNSLATHTENRATRLIDLNQGDTMLIEGQYRPCPRCKGAMRVKAEESGAKVIYTWPEDGDLKKREWEGTPCDKK

SEQ ID NO: 11 - DNA, 354 bp, synthetic construct
  misc_feature 1..354: note = BadTF3I encoding sequence
  source 1..354: mol_type = other DNA
atgaccaaatctaaaatgctgagcaacatcgtcatccaggaggtcaaatttgcgatcgaagattactgcgctattctgagcttcgcttctgactcttatgaagtgccggagcagtattttatcattacccgttctaccaccgaacgttctggcggtattccggagggcgacatctacctggaatctaacctgtttctggattttaacccgtacggcctgagcggttacctgctgtctgagccgaactgcgtagatctgctgatcgaaccgaacaactacgttcgtctgcgtctgatcgaaaaaatcgatatcctggaagtggaaaaccacctgaaatttctgttcgacaactaa

SEQ ID NO: 12 - protein, 117 aa, synthetic construct
  REGION 1..117: note = BadTF3I encoding sequence
MTKSKMLSNIVIQEVKFAIEDYCAILSFASDSYEVPEQYFIITRSTTERSGGIPEGDIYLESNLFLDFNPYGLSGYLLSEPNCVDLLIEPNNYVRLRLIEKIDILEVENHLKFLFDN

SEQ ID NO: 13 - protein, 152 aa, synthetic construct
  REGION 1..152: note = DddA11 with N terminal
MGSSHHHHHHSQDPGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAKMTVVPPEGAIPVKRGATGETKVFIGNSNSPKSPTKGGC

SEQ ID NO: 14 - protein, 121 aa, synthetic construct
  REGION 1..121: note = BadTF3
GWKFSNGKRRPPHKATVTVTDKNGVVKHKSNLVSGNMTEAEKKLGFPNNSLATHTENRATRLIDLNQGDTMLIEGQYRPCPRCKGAMRVKAEESGAKVIYTWPEDGDLKKREWEGTPCDKK

SEQ ID NO: 15 - protein, 138 aa, synthetic construct
  REGION 1..138: note = DddAtox
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

(19) United States
(12) Patent Application Publication
(10) Pub. No.: US 2026/0337664 A1
(43) Pub. Date: Oct. 29, 2026

(54) METHODS OF DETERMINING GENOME-WIDE DNA BINDING PROTEIN BINDING SITES BY FOOTPRINTING WITH DOUBLE STRANDED DNA DEAMINASE

(71) Applicants: BEIJING CHANGPING LABORATORY, Beijing (CN); PEKING UNIVERSITY, Bejing (CN)

(72) Inventors: Wenyang Dong, Beijing (CN); Runsheng He, Beijing (CN); Zhi Wang, Beijing (CN); Xiaoliang Xie, Beijing (CN)

(73) Assignees: BEIJING CHANGPING LABORATORY, Beijing (CN); PEKING UNIVERSITY, Beijing (CN)

(21) Appl. No.: 19/115,056

(22) PCT Filed: Sep. 30, 2022
(86) PCT No.: PCT/CN2022/123373

§ 371 Date: Mar. 25, 2025

(51) Int. Cl. C12Q 1/6827; C12N 9/78; C12N 15/52

(52) U.S. Cl. CPC C12Q 1/6827; C12N 9/78; C12N 15/52; C12Y 305/04001

(57) ABSTRACT

Methods are provided for determining a transcription factor binding site on genomic double stranded (ds) DNA of a eukaryotic cell or cells by contacting the genomic dsDNA with a dsDNA deaminase under conditions to convert cytosine of the genomic dsDNA to uracil, thereby creating treated genomic dsDNA, and identifying unconverted cytosine on the treated genomic dsDNA as a transcription factor binding site.

### FIELD

The present disclosure generally relates to systems, methods and compositions for determining DNA binding protein binding sites along the genome in a cell or cells.

### BACKGROUND

Each cell of an individual has essentially the same genome, yet they carry out completely different functions in each tissue. The advent of single cell genomics has allowed determination of the transcriptome, methylome and open chromosome sites of a single human cell, which allows categorization of cell types in unprecedented ways. However, beyond cell typing, the compelling challenge is decoding the human functional genome, i.e. understanding cell functions based on the human genome. Processes such as gene expression and regulation, cell differentiation and development, are pertinent to chromatin structures, and regulatory networks, for which transcription factors (TF) are of critical importance.

There are only about 1000 TFs in humans, controlling about 20,000 genes. The specificity of gene regulation is achieved through a combinatorial binding of several TFs, which act like a keyset to turn on and off a particular gene. Therefore, it is important to learn about the precise binding sets of TFs and how they cooperate with each other. Current methods for detecting DNA protein interaction at a genome-wide level, such as ChIP-seq, have several problems including high signal/noise ratio, low through-put, low resolution, and requirements for cell number and homogeneity. Importantly, existing methods for detecting DNA protein interaction at a genome-wide level lack the ability to identify TFs that cooperate with each other during gene regulation.

### SEQUENCE LISTING

The instant application contains a Sequence Listing which has been submitted electronically in XML format and is hereby incorporated by reference in its entirety. Said XML copy, created on Sep. 28, 2022, is named “009191.00002_st26” and is 23 KB in size.

### SUMMARY

In general, aspects of the present disclosure are directed to methods for identifying or profiling DNA binding proteins, such as transcription factors, on double stranded polynucleotides such as genomic DNA. In general, methods of the present disclosure utilize a double stranded (ds) DNA deaminase to convert cytosine to uracil on the double stranded polynucleotide, except where a DNA binding protein is bound to the polynucleotide. The dsDNA deaminase is sterically prevented from converting cytosine to uracil at the location where the DNA binding protein is bound to the polynucleotide, thereby creating a “footprint” where cytosine was not converted to uracil. Accordingly, the location where the DNA binding protein is bound to the polynucleotide can be determined. The determined DNA binding site can be compared to known DNA binding sites of DNA binding proteins to thereby identify the DNA binding protein that was bound to the determined DNA binding site. Using the methods described herein, one or more or a plurality of DNA binding proteins can be identified for a given polynucleotide, such as a gene within chromatin DNA.

Aspects of the present disclosure are directed to methods of identifying binding sites of one or more transcription factors (TF) to DNA, such as chromatin DNA. Identification of the binding sites of transcription factors can be used to identify the transcription factors themselves based on their known binding sites with chromatin DNA, and accordingly, the one or more, or pairs of, or plurality of transcription factors that cooperate to regulate a gene. According to one aspect, transcription factor combinations or “keysets” are decoded for a particular gene, and along the genome, thereby identifying transcription factor combinations or keysets genome wide.

According to one aspect, a method is provided including contacting chromatin DNA with a dsDNA deaminase. The dsDNA deaminase converts cytosine to uracil along the chromatin DNA unless a TF is bound to the chromatin DNA. The binding of the TF to chromatin DNA sterically prevents the dsDNA deaminase from converting cytosine to uracil at the binding site between the TF and the chromatin DNA. Accordingly, conversion of cytosine to uracil occurs on either side of the binding site between the TF and the chromatin DNA. The boundary of where TF binds with chromatin DNA based on cytosine to uracil conversion can be determined, and accordingly a “footprint” of the binding site is determined. The binding site is then compared with known binding sites of TFs to identify the TF with the matching binding site. Target chromatin DNA, such as a gene, can be analyzed to determine whether one or more, or a pair, or a plurality of TFs bind to the target chromatin DNA, allowing identification of TFs involved in regulation of a particular gene. According to one aspect, this approach of identifying TF keysets may be implemented genome wide across all genes.

In general, aspects of the present disclosure include cell permeabilization or nuclei permeabilization and isolation of a cell, single cell or population of cells. In this manner, the genomic DNA of the cell, single cell or populations of cells is made more accessible to a dsDNA deaminase. According to one aspect, a cell or cells or nucleus or nuclei need not be permeabilized while still allowing treatment with a dsDNA deaminase. Other methods of treating a cell or cells or a nucleus or nuclei to make a cell or cells or nucleus or nuclei more accessible to treatment with a dsDNA deaminase according to known methods such as lysis are contemplated.

According to one aspect, the permeabilized cell or cells or permeabilized nucleus or nuclei may be treated with a crosslinking agent to crosslink cellular components to maintain cellular structure as is known in the art prior to treatment with a double stranded DNA deaminase. According to one aspect, a double stranded polynucleotide molecule that has DNA binding proteins bound to it, such as in the case of cell-free DNA, may be treated with a dsDNA deaminase.

According to one aspect, the permeabilized cell or cells or permeabilized nucleus or nuclei or cell-free DNA are treated with the double stranded DNA deaminase in a manner to convert cytosine to uracil in DNA of the cell or cells or nucleus or nuclei by hydrolysis removal of an amino group from cytosine nucleotides available for deamination to create uracil nucleotides. According to one aspect, DNA may be fragmented and enriched prior to treatment with a dsDNA deaminase. According to one aspect, DNA may be treated with a dsDNA deaminase and then fragmented and enriched. Treatment with a dsDNA deaminase results in treated DNA, insofar as the treated DNA includes one or more uracils resulting from the treatment with a dsDNA deaminase. Exemplary double stranded DNA deaminases include double stranded DNA deaminase A (“DddA”) known in the art, an evolved double stranded DNA deaminase A11 (“DddA11”) known in the art and bacterial deaminase toxin family 3 (“BadTF3”) known in the art. The treated DNA, such as treated chromatin DNA may be processed using transposases or DNase to enrich for open chromatin DNA, i.e. transcriptionally active genomic DNA that can be accessed by DNA regulatory elements.

The treated DNA can be amplified prior to sequencing. Exemplary amplification methods include PCR. Alternatively, the treated DNA for sequencing can be sequenced directly after library preparation without amplification.

The treated DNA can be sequenced. According to one aspect, the treated DNA can be sequenced as whole genome DNA. According to one aspect, the treated DNA can be sequenced as accessible regions of chromatin through cleavage by enzymes such as a transposase or a nuclease such as DNase, MNase or a restriction endonuclease. According to one aspect, enriched open chromatin DNA is sequenced to determine cytosine to uracil conversion, and accordingly, the DNA binding protein footprint.

According to one aspect, a targeted chromatin region, such as an open chromatin region, may be enriched before or after treatment with a dsDNA deaminase using methods known to those of skill in the art including ChIP-seq, ChIC, ChEC, ChEC-seq, CUT&TAG, CUT&RUN, Multi-CUT&Tag, NTT-seq, R loop CUT&Tag and the like, in addition to methods that use a transposase, such as Tn5 transposase. For example, dsDNA is treated with a dsDNA deaminase and then the treated DNA is enriched for open chromatin regions for sequencing using CUT&TAG. Alternatively, dsDNA is enriched for open chromatin regions for sequencing using CUT&TAG, and the enriched DNA is then treated with a dsDNA deaminase.

According to one aspect, a library can be prepared based on whole genome DNA as is known in the art. According to one aspect, a library can be prepared based on DNA regions of interest, such as open chromatin. According to one aspect, the targeted region is enriched by using antibodies in methods such as ChIP-seq, ChIC, ChEC, ChEC-seq, CUT&TAG, and CUT&RUN. According to one aspect, the target region is enriched using other binding agents, such as a nanobody (see Stuart et al., Nanobody-tethered transposition allows for multifactorial chromatin profiling at single-cell resolution, bioRxiv 10.1101/2022.03.08.483436v1 hereby incorporated by reference in its entirety) or a specific chromatin binding domain (see Wang et al., Genomic profiling of native R loops with a DNA-RNA hybrid recognition sensor, Sci. Adv. 2021 February; 7(8) eabe3516 10.1126/sciadv.abe3516 hereby incorporated by reference in its entirety.)

DNA binding proteins can then be profiled by analyzing information obtained from the cytosine to uracil conversion sites on sequence reads. A nonconverted site (i.e., a cytosine remains a cytosine during treatment with a dsDNA deaminase) indicates a binding site where a DNA binding protein was bound thereto at the time of treatment with a dsDNA deaminase. A converted site (i.e., cytosine converted to uracil by a dsDNA deaminase) indicates a site where a DNA binding protein was not bound thereto at the time of treatment with a dsDNA deaminase. DNA binding protein binding profiles can be compared to the nonconverted site or sites using methods known to those of skill to identify a particular DNA binding protein using, for example a database of binding sites associated with DNA binding proteins, such as JASPAR (world wide website jaspar.genereg.net), CIS-BP (world wide website cisbp.ccbr.utoronto.ca), HOCOMOCO (world side website hocomocol1.autosome.org) which are TF motif databases. Potential binding TFs are identified by comparing the footprints identified by the dsDNA deaminase methods described herein with the known TF motifs from these databases.

Aspects of the present disclosure may be carried out on a single cell level or single DNA molecule level or with a plurality of cells. The plurality of cells may be of the same cell type. The plurality of cells may be of different cell type.

According to the present disclosure, methods are provided to quantify the simultaneous binding of multiple TFs on single DNA molecules. The methods provide high-resolution binding maps of multiple TFs at a genome-wide level. According to the present disclosure, methods are provided to analyze how TFs cooperate or antagonize during the regulation of transcription.

According to one aspect, use of a dsDNA deaminase in manufacture of an agent for carrying out the method of determining a transcription factor binding site on genomic double stranded (ds) DNA of a eukaryotic cell is provided. In certain embodiments, the method is as described herein. In certain embodiments, the method comprises contacting the genomic dsDNA with the dsDNA deaminase under conditions to convert cytosine of the genomic dsDNA to uracil, thereby creating treated genomic dsDNA, and identifying unconverted cytosine on the treated genomic dsDNA as a transcription factor binding site.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawing in which:

FIG. 1 is a schematic depicting determination of TF binding sites using a double stranded DNA deaminase.

FIG. 2 depicts a vector map of pETDuet-1::dddAtox+dddAI.

FIG. 3 depicts a vector map of pETDuet-1::badTF3tox+badTF3I.

FIG. 4 depicts a vector map of pETDuet-1::dddA11+dddAI

FIG. 5 is an SDS-PAGE gel stained with Coomassie blue of DddAtox-DddAI, DddAtox, DddA11-DddAI, DddA11, BadTF3-BadTF3I and BadTF3, respectively.

FIG. 6 depicts data supporting the conversion ratio of cytosine to uracil using several double stranded DNA deaminases.

FIGS. 7A and 7B depict data identifying TF footprints. FIG. 7C depicts Venn diagrams displaying the overlap between CTCF binding identified by the dsDNA deaminase method described herein, a ChIP-seq method and a DNase-seq method.

FIG. 8A is a schematic depicting TF binding patterns on a single molecule level. FIG. 8B depicts data demonstrating identification and quantification of the binding of the transcription factor CTCF to DNA. FIG. 8C depicts data demonstrating the use of the dsDNA deaminase method described herein to identify multiple transcription factor binding sites.

FIG. 9A is a schematic depicting one aspect of the method of the present disclosure. FIG. 9B depicts DNA fragment distribution data generated by methods described herein. FIG. 9C depicts cell typing results of single cell data generated by methods described herein for K562, GM12878 and HEK293T cell lines. FIG. 9D depicts data a comparison of bulk and single cell data generated by methods described herein viewed by IGV software.

### DETAILED DESCRIPTION

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

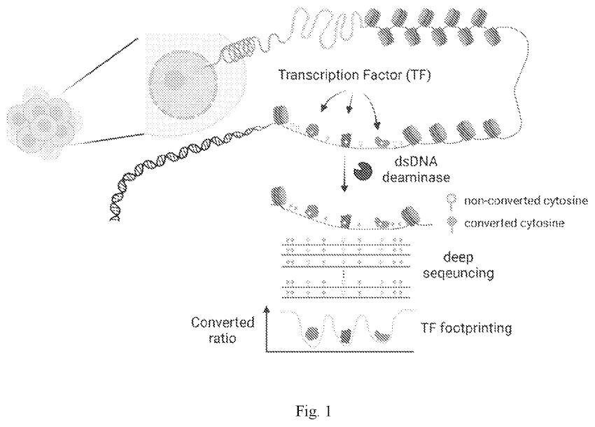

Aspects of the present disclosure are described with reference to FIG. 1. As shown in FIG. 1, nuclei are extracted from cells, cell lines or tissues and permeabilized. The nuclei include chromatin DNA with DNA binding proteins, such as TFs, bound thereto. Isolated permeabilized nuclei are incubated, i.e. treated, with a dsDNA cytosine deaminase that converts accessible cytosine to uracil on dsDNA to produce treated DNA, such as treated chromatin DNA. Accessible cytosine is cytosine that is not sterically hindered, i.e. sterically unhindered, from enzymatic reaction with the dsDNA cytosine deaminase. Inaccessible cytosine is sterically hindered cytosine bound by a DNA binding protein such as a TF, and is therefor inaccessible or otherwise unavailable for enzymatic reaction with the dsDNA cytosine deaminase. Regions of chromatin DNA not bound by a DNA binding protein such as regions of chromatin DNA immediately adjacent to and flanking a DNA binding protein (“flanking regions”) are accessible to dsDNA deaminases which convert cytosine to uracil in the regions of chromatin DNA not bound to a DNA binding protein. Regions of chromatin DNA bound by a DNA binding protein or DNA binding proteins, such as transcription factors or other DNA binding proteins, are protected from cytosine to uracil conversion by the dsDNA deaminase. As a result, regions of conversion are adjacent to or otherwise flank regions of nonconversion, with the regions of nonconversion corresponding to the DNA binding protein binding site. Such a DNA binding protein binding site is referred to as a DNA binding protein “footprint”. The DNA binding protein binding site may be a single nucleotide or be one or more nucleotides, two or more nucleotides, three or more nucleotides, four or more nucleotides, be from 1 to 4 or 1 to 5 nucleotides or extend over various nucleotides or otherwise be various nucleotides in length. The DNA binding protein binding site may be a single nucleotide or a plurality of nucleotides. The DNA binding protein may interact with one or more nucleotides of the chromatin DNA. The nucleotides that interact with the DNA binding protein may define the DNA protein binding site. According to one aspect, treated chromatin DNA is extracted for whole-genome or targeted amplicon sequencing. The methods described herein allow the simultaneous quantification of multiple DNA binding protein binding events (such as TF binding events whether pairs of TFs or more than two TFs, three TFs, four TFs, five TFs, etc.) on a gene of a single DNA molecule. Methods described herein are directed to systematically quantify the frequency of co-occupancy for a plurality of TFs or pairs of TFs, such as thousands of TFs or pairs of TFs, across multiple genes across the genome.

### Cells

Cells according to the invention include any cell where understanding DNA binding protein binding sites for double stranded DNA, such as chromatin DNA, is considered by those of skill in the art to be useful. Cells include prokaryotic cells or eukaryotic cells. A cell according to the present disclosure includes a cancer cell of any type, hepatocyte, oocyte, embryo, stem cell, iPS cell, ES cell, neuron, erythrocyte, melanocyte, astrocyte, germ cell, oligodendrocyte, kidney cell and the like.

Cells useful in the methods described herein can be obtained from a biological sample, tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, such as a population of prokaryotic or eukaryotic single celled organisms including bacteria or yeast. According to one aspect, the sample may be in vitro. The term “in vitro” has its art recognized meaning, e.g., involving purified reagents or extracts, e.g., cell extracts. As used herein, the term “biological sample” is intended to include, but is not limited to, tissues, cells, biological fluids and isolates thereof, isolated from a subject, as well as tissues, cells and fluids present within a subject.

According to one aspect, the methods of the present invention are practiced with a single cell. As used herein, a “single cell” refers to one cell. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example, a 96-well plate, such that each single cell is placed in a single well.

Methods for manipulating single cells are known in the art and include fluorescence activated cell sorting (FACS), flow cytometry (Herzenberg., PNAS USA 76:1453-55 1979), micromanipulation and the use of semi-automated cell pickers (e.g. the QUIXELL cell transfer system from Stoelting Co.). Individual cells can, for example, be individually selected based on features detectable by microscopic observation, such as location, morphology, or reporter gene expression. Additionally, a combination of gradient centrifugation and flow cytometry can also be used to increase isolation or sorting efficiency.

According to one aspect, the methods of the present invention are practiced with a plurality of cells. A plurality of cells includes from about 2 to about 1,000,000 cells, about 2 to about 10 cells, about 2 to about 100 cells, about 2 to about 1,000 cells, about 2 to about 10,000 cells, about 2 to about 100,000 cells, about 2 to about 10 cells or about 2 to about 5 cells.

According to one aspect, the DNA to be treated is genomic DNA or chromatin DNA. According to one aspect, the DNA to be treated is mammalian DNA, plant DNA, yeast DNA, viral DNA, or prokaryotic DNA. In yet another preferred embodiment, the DNA sample is obtained from a human, bovine, porcine, ovine, equine, rodent, avian, fish, shrimp, plant, yeast, virus, or bacteria. Preferably the DNA to be treated is genomic DNA. The term “genome” as used herein is defined as the collective gene set carried by an individual, cell, or organelle. The term “genomic DNA” as used herein is defined as DNA material comprising the partial or full collective gene set carried by an individual, cell, or organelle.

According to one aspect, the DNA to be treated is a double stranded polynucleotide molecule with protein binding thereto, such as cell-free DNA. According to this aspect, DNA, such as genomic DNA or chromatin DNA, may be isolated and treated with a dsDNA deaminase and processed and analysed as described herein.

### Methods of Permeabilization of Cell or Nucleus

Once a desired cell or cells have been identified, the methods described herein may be practiced on the cell or cells or a nucleus or nuclei obtained from the cell or cells. An individual cell or plurality of cells or nucleus or nuclei may be isolated. The cell or cells or nucleus or nuclei may be treated according to known methods to facilitate entry of chemicals, drugs, enzymes such as a dsDNA deaminase, DNA or other reagents to be introduced into the cell or cells or nucleus or nuclei.

According to one aspect, the cell or cells or a nucleus or nuclei may be permeabilized. Methods of permeabilization are known to those of skill in the art. Exemplary permeabilization techniques include electroporation or electropermeabilization, permeabilization with mild non-ionic detergents such as saponin and digitonin and by pore-forming toxins, such as alpha-toxin and streptolysin O, as is known in the art. Electroporation, or electropermeabilization or electrotransfer, is a technique in which an electrical field is applied to cells or the nuclei of cells in order to increase the permeability of the cell membrane, allowing chemicals, drugs, enzymes such as a dsDNA deaminase, electrode arrays or DNA to be introduced into the cell.

Alternatively, the cell or cells may be lysed to obtain the nucleus or nuclei, or the nucleus or nuclei may be lysed, using methods known to those of skill in the art. Lysis can be achieved by, for example, heating the cells, or by the use of detergents or other chemical methods, or by a combination of these. However, any suitable lysis method known in the art can be used.

Alternatively, DNA, such as genomic DNA or chromatin DNA, may be extracted or otherwise isolated from a tissue sample, blood sample, cell or cells and the like according to methods known in the art. DNA extraction protocols using beads (such as DYNABEADS) or reagents are known to those of skill and are commercially available in kits through ThermoFisher Scientific, for example CHARGESWITCH genomic DNA purification kits, and the like.

### Methods of Crosslinking Cells and Nuclei

In certain embodiments, the cell or cell or nucleus or nuclei are treated with a crosslinking agent to maintain cellular structure as is known in the art before dsDNA deaminase treatment. Such crosslinking treatments include treatment with paraformaldehyde or treatment with ultra violet light to effect crosslinking. The crosslinking is used to maintain cellular structure, and in some cases the binding TF may be stably crosslinked with the DNA. Methods described herein can be applied to cells and nuclei treated with a crosslinking agent. According to one aspect, methods described herein can be applied to Formalin-Fixed and Paraffin-Embedded (FFPE) samples and the like, as is known in the art. For example, FFPE is a form of preservation and preparation of specimens. A tissue sample is first preserved by fixing it in formaldehyde, also known as formalin, such as a solution of 10% neutral-buffered formalin for about 18-24 hours, to preserve the proteins and vital structures within the tissue. Next, it is embedded in a paraffin wax block, samples of which can then be processed according to the methods described herein. To prepare for infiltration by wax, the tissue may be dehydrated and cleared, often using increasing concentrates of ethanol. Then, it is embedded into IHC-grade paraffin according to known methods.

### DNA Binding Proteins

According to certain aspects of the present disclosure, methods are provided to determine the DNA binding protein binding site on DNA, such as chromatin DNA or a cell. DNA-binding proteins are proteins that have DNA-binding domains and thus have a specific or general affinity for single- or double-stranded DNA. Sequence-specific DNA-binding proteins generally interact with the major groove of B-DNA, because it exposes more functional groups that identify a base pair.

DNA-binding proteins include transcription factors which modulate the process of transcription, various polymerases, nucleases which cleave DNA molecules, and histones which are involved in chromosome packaging and transcription in the cell nucleus. DNA-binding proteins can incorporate such domains as the zinc finger, the helix-turn-helix, and the leucine zipper (among many others) that facilitate binding to nucleic acid.

Structural proteins that bind DNA are well-understood examples of non-specific DNA-protein interactions. Within chromosomes, DNA is held in complexes with structural proteins. These proteins organize the DNA into a compact structure called chromatin. In eukaryotes, this structure involves DNA binding to a complex of small basic proteins called histones. The histones form a disk-shaped complex called a nucleosome, which contains two complete turns of double-stranded DNA wrapped around its surface. These non-specific interactions are formed through basic residues in the histones making ionic bonds to the acidic sugar-phosphate backbone of the DNA, and are therefore largely independent of the base sequence. Chemical modifications of these basic amino acid residues include methylation, phosphorylation and acetylation. These chemical changes alter the strength of the interaction between the DNA and the histones, making the DNA more or less accessible to transcription factors and changing the rate of transcription. Other non-specific DNA-binding proteins in chromatin include the high-mobility group (HMG) proteins, which bind to bent or distorted DNA. These proteins are important in bending arrays of nucleosomes and arranging them into the larger structures that form chromosomes.

In contrast, transcription factors bind to specific DNA sequences. As is known in the art, each transcription factor binds to one specific set of DNA sequences and activates or inhibits the transcription of genes that have these sequences near their promoters. The specificity of transcription factors' interactions with DNA come from the proteins making multiple contacts to the edges of the DNA bases, allowing them to read the DNA sequence. Most of these base-interactions are made in the major groove, where the bases are most accessible. A transcription factor (TF) (or sequence-specific DNA-binding factor) is a protein that controls the rate of transcription of genetic information from DNA to messenger RNA, by binding to a specific DNA sequence. The function of TFs is to regulate-turn on and off-genes in order to make sure that they are expressed in the desired cells at the right time and in the right amount throughout the life of the cell and the organism. Groups of TFs function in a coordinated fashion to direct cell division, cell growth, and cell death throughout life; cell migration and organization (body plan) during embryonic development; and intermittently in response to signals from outside the cell, such as a hormone. There are up to 1600 TFs in the human genome. See Babu M M, Luscombe N M, Aravind L, Gerstein M, Teichmann S A (June 2004). “Structure and evolution of transcriptional regulatory networks” (PDF). Current Opinion in Structural Biology. 14 (3): 283-91. doi:10.1016/j.sbi.2004.05.004. PMID 15193307 hereby incorporated by reference in its entirety.

TFs work alone or with other proteins in a complex, by promoting (as an activator), or blocking (as a repressor) the recruitment of RNA polymerase (the enzyme that performs the transcription of genetic information from DNA to RNA) to specific genes. A defining feature of TFs is that they contain at least one DNA-binding domain (DBD), which attaches to a specific sequence of DNA adjacent to the genes that they regulate. See Mitchell P J, Tjian R (July 1989). “Transcriptional regulation in mammalian cells by sequence-specific DNA binding proteins”. Science. 245 (4916): 371-8. Bibcode:1989Sci . . . 245 . . . 371M. doi:10.1126/science.2667136. PMID 2667136; Ptashne M, Gann A (April 1997). “Transcriptional activation by recruitment”. Nature. 386 (6625): 569-77. Bibcode:1997Natur.386 . . . 569P. doi:10.1038/386569a0. PMID 9121580. S2CID 6203915. each of which are hereby incorporated by reference in its entirety for the teaching of known transcription factors. TFs are grouped into classes based on their DNA-binding domains. See Stegmaier P, Kel A E, Wingender E (2004). “Systematic DNA-binding domain classification of transcription factors”. Genome Informatics. International Conference on Genome Informatics. 15 (2): 276-86. PMID 15706513. Archived from the original on 19 Jun. 2013; Matys V, et al. (January 2006). “TRANSFAC and its module TRANSCompel: transcriptional gene regulation in eukaryotes”. Nucleic Acids Research. 34 (Database issue): D108-10. doi:10.1093/nar/gkj143. PMC 1347505. PMID 16381825 each of which are hereby incorporated by reference in its entirety for its teaching of transcription factors and their associated DNAS-binding domains.

According to one aspect, exemplary transcription factors include but are not limited to AAF, ABL, ADA2, ADANF1, AF1, AFP1, AHR, AIIN3, AIRE, ALL1, ALPHACBF, ALPHACP1, ALPHACP2A, ALPHACP2B, ALPHAH2, ALPHAH3, ALPHAHO, ALX1, ALX3, ALX4, AMEF2, AML1, AML1A, AML1B, AML1C, AMLlDELTAN, AML2, AML3, AML3A, AML3B, AMY1L, AMYB, ANF, ANHX, AP1, AP2ALPHAA, AP2ALPHAB, AP2BETA, AP2GAMMA, AP3(1), AP3(2), AP4, AP5, APC, AR, AREB6, ARGFX, ARID5B, ARNT, ARNT(774MFORM), ARNT2, ARNT::HIF1A, ARNTL, ARP1, ARX, ASCL1, ASCL2, ATBF1A, ATBF1B, ATF, ATF1, ATF2, ATF3, ATF3DELTAZIP, ATF4, ATF6, ATF6B, ATF7, ATFA, ATFADELTA, ATOH1, ATOH7, ATPF1, B, BACH1, BACH2, BANP, BARH11, BARH12, BARHL1, BARHL2, BARX1, BARX2, BATF, BATF3, BATF::JUN, BBX, BCL11A, BCL11B, BCL3, BCL6, BCL6B, BD73, BETACATENIN, BHLHA15, BHLHE22, BHLHE23, BHLHE40, BHLHE41, BIN1, BMYB, BNC2, BP1, BP2, BPTF, BRAHMA, BRCA1, BRN3A, BRN3B, BRN4, BSX, BTEB, BTEB2, BTFIID, C/EBPALPHA, C/EBPBETA, C/EBPDELTA, CACCBINDINGFACTOR, CART1, CBF(4), CBF(5), CBP, CCAATBINDINGFACTOR, CCF, CCG1, CCK1A, CCK1B, CCMTBINDINGFACTOR, CD28RC, CDC5L, CDK2, CDK9, CDX1, CDX2, CDX4, CEBPA, CEBPB, CEBPD, CEBPE, CEBPG, CENPB, CENPBD1, CFF, CHXLO, CLIM2, CLIM1, CLOCK, CNBP, COS, COUP, CP1, CP2, CPBP, CPEB1, CPEBINDINGPROTEIN, CPIA, CPIC, CREB, CREB1, CREB2, CREB3, CREB3L1, CREB3L4, CREB5, CREBPLCREBPA, CREM, CREMALPHA, CRF, CRX, CSBP1, CTCF, CTCFL, CTF, CTF1, CTF2, CTF3, CTF5, CTF7, CUP, CUTL1, CUX1, CUX2, CX, CXXC5, CYCLINA, CYCLINT1, CYCLINT2, CYCLINT2A, CYCLINT2B, DAP, DAX1, DB1, DBF4, DBP, DBPA, DBPAV, DBPB, DDB, DDB1, DDB2, DEF, DELTACREB, DELTAMAX, DF1, DF2, DF3, DIX4(LONGISOFORM), DLX1, DLX2, DLX3, DLX4, DLX4(SHORTISOFORM, DLX5, DLX6, DMRT1, DMRT2, DMRT3, DMRTA1, DMRTA2, DMRTC2, DNMT1, DP1, DP2, DPF1, DPRX, DRGX, DSIF, DSIFP14, DSIFP160, DTF, DUX1, DUX2, DUX3, DUX4, DUXA, E, E12, E2F, E2F+E4, E2F+P107, E2F1, E2F2, E2F3, E2F4, E2F5, E2F6, E2F7, E2F8, E47, E4BP4, E4F, E4F1, E4TF2, EAR2, EBF1, EBF3, EBP80, EC2, EF1, EFC, EGR1, EGR2, EGR3, EGR4, EHF, EIF1, EIIAEA, EIIAEB, EIIAECALPHA, EIIAECBETA, EIVF, ELF1, ELF2, ELF3, ELF4, ELF5, ELK1, ELK1::HOXA1, ELK1::HOXB13, ELK1::SREBF2, ELK3, ELK4, EMX1, EMX2, EN1, EN2, ENHBIND.PROT, ENKTF1, EOMES, EPAS1, EPSILONF1, ER, ERF, ERF::FIGLA, ERF::FOXI1, ERF::FOXO1, ERF::HOXB13, ERF::NHLH1, ERF::SREBF2, ERG, ERG1, ERG2, ERR1, ERR2, ESR1, ESR2, ESRRA, ESRRB, ESRRG, ESX1, ETF, ETS1, ETS1DELTAVIL, ETS2, ETV1, ETV2, ETV2::DRGX, ETV2::FIGLA, ETV2::FOXI1, ETV2::HOXB13, ETV3, ETV4, ETV5, ETV5::DRGX, ETV5::FIGLA, ETV5::FOXI1, ETV5::FOXO1, ETV5::HOXA2, ETV6, ETV7, EVX1, EVX2, F2F, FACTOR2, FACTORNAME, FBP, FEBP, FERD3L, FEV, FEZF1, FIGLA, FKBP59, FKHL18, FKHRL1P2, FLI1, FLI1::DRGX, FLI1::FOXI1, FOS, FOS::JUN, FOS::JUNB, FOS::JUND, FOSB, FOSB::JUN, FOSB:: JUNB, FOSL1, FOSL1 JUN, FOSL1::JUNB, FOSL1::JUND, FOSL2, FOSL2::JUN, FOSL2::JUNB, FOSL2::JUND, FOXA1, FOXA2, FOXA3, FOXB1, FOXC1, FOXC2, FOXD1, FOXD2, FOXD3, FOXD4, FOXE1, FOXE3, FOXF1, FOXF2, FOXG1, FOXG1A, FOXG1B, FOXG1C, FOXH1, FOXI1, FOXJ1A, FOXJ1B, FOXJ2, FOXJ2(LONGISOFORM), FOXJ2(SHORTISOFORM), FOXJ2::ELF1, FOXJ3, FOXK1, FOXK1A, FOXK1B, FOXK1C, FOXK2, FOXL1, FOXL2, FOXM1, FOXM1A, FOXM1B, FOXM1C, FOXN1, FOXN2, FOXN3, FOXO1, FOXO1::ELF1, FOXO1::ELK1, FOXO1::ELK3, FOXO1::FLI1, FOXO1A, FOXO1B, FOXO2, FOXO3, FOXO3A, FOXO3B, FOXO4, FOXO6, FOXP1, FOXP2, FOXP3, FOXQ1, FOXR1, FOXR2, FRA1, FRA2, FTF, FTS, G6FACTOR, GABP, GABPA, GABPALPHA, GABPBETA1, GABPBETA2, GADD153, GAF, GAMMACAC1, GAMMACAC2, GAMMACMT, GATA1, GATA1::TAL1, GATA2, GATA3, GATA4, GATA5, GATA6, GBX1, GBX2, GCF, GCM1, GCM2, GCMA, GCNS, GF1, GFACTOR, GFI1, GFI1B, GLI, GLI1, GLI2, GLI3, GLI4, GLIS1, GLIS2, GLIS3, GMEB1, GMEB2, GRALPHA, GRBETA, GRF1, GRHL1, GRHL2, GSC, GSC2, GSCL, GSX1, GSX2, GTF3A, GTIC, GTIIA, GTIIBALPHA, GTIIBBETA, H1TF1, H1TF2, H2RIIBP, H4TF1, H4TF2, HAND1, HAND2, HB9, HDAC1, HDAC2, HDAC3, HDAXX, HDX, HEATINDUCEDFACTOR, HEB, HEB1P67, HEB1P94, HEF1B, HEFIT, HEF4C, HEN1, HEN2, HES1, HES2, HES5, HES6, HES7, HESX1, HEX, HEYl, HEY2, HIC1, HIC2, HIF1, HIF1A, HIF1ALPHA, HIF1BETA, HINFA, HINFB, HINFC, HINFD, HINFD3, HINFE, HINFP, HIP1, HIVEP2, HKR1, HLF, HLTF, HLTF(MET123), HLX, HMBOX1, HMBP, HMGI, HMGI(Y), HMGIC, HMGY, HMX1, HMX2, HMX3, HNF1A, HNF1B, HNF3, HNF3ALPHA, HNF3BETA, HNF3GAMMA, HNF4, HNF4A, HNF4ALPHA, HNF4ALPHA1, HNF4ALPHA2, HNF4ALPHA3, HNF4ALPHA4, HNF4G, HNF4GAMMA, HNF6ALPHA, HNFIA, HNFIB, HNFIC, HNRNPK, HOMEZ, HOX11, HOXA1, HOXA10, HOXA11, HOXA13, HOXA2, HOXA3, HOXA4, HOXA5, HOXA6, HOXA7, HOXA9, HOXA9A, HOXA9B, HOXAIO, HOXAIOPL2, HOXB1, HOXB13, HOXB2, HOXB2::ELK1, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD12::ELK1, HOXD13, HOXD3, HOXD4, HOXD8, HOXD9, HP55, HP65, HPX42B, HRPF, HSF, HSF1, HSF1(LONG), HSF1(SHORT), HSF2, HSF4, HSF5, HSFY1, HSFY2, HSP56, HSP90, IBP1, ICERII, ICERLIGAMMA, ICSBP, ID1, ID1H′, ID2, ID3, ID3/HEIR1, IF1, IGPE1, IGPE2, IGPE3, II1RF, IKAPPAB, IKAPPABALPHA, IKAPPABBETA, IKAPPABR, IKZF1, IKZF3, IL6REBP, INSAF, INSM1, IPF1, IRF1, IRF2, IRF3, IRF4, IRF5, IRF6, IRF7, IRF8, IRF9, IRX1, IRX2, IRX2A, IRX3, IRX4, IRX5, ISGF1, ISGF3, ISGF3ALPHA, ISGF3GAMMA, ISL1, ISL2, ISX, ITF, ITF1, ITF2, JDP2, JRF, JUN, JUN::JUNB, JUNB, JUND, KAPPAYFACTOR, KBP1, KDM2B, KERI, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KOX1, KRF1, KUAUTOANTIGEN, KUP, LBP1, LBP1A, LBX1, LBX2, LCORL, LCRF1, LEF1, LEFIB, LFA1, LHX1, LHX2, LHX3, LHX3A, LHX3B, LHX5, LHX6, LHX6.1A, LHX6.1B, LHX8, LHX9, LIN28B, LIT1, LMO1, LMO2, LMX1A, LMX1B, LMY1(LONGFORM), LMY1(SHORTFORM), LMY2, LSF, LXRALPHA, LY 11, LYF1, LYL1, MAD1, MAF, MAF::NFE2, MAFA, MAFB, MAFF, MAFG, MAFG::NFE2L1, MAFK, MASH1, MAX, MAXi, MAX2, MAX::MYC, MAZ, MAZ1, MB67, MBD2, MBF1, MBF2, MBF3, MBNL2, MBP1(1), MBP1(2), MBP2, MDBP, MECOM, MECP2, MEF2, MEF2A, MEF2B, MEF2C, MEF2C(433AAFORM), MEF2C(465AAFORM), MEF2C(473MFORM), MEF2C/DELTA32(441AAFORM), MEF2D, MEF2D00, MEF2D0B, MEF2DA′B, MEF2DA0, MEF2DAB, MEF2DAO, MEIS1, MEIS2, MEIS2A, MEIS2B, MEIS2C, MEIS2D, MEIS2E, MEIS3, MEOX1, MEOX1A, MEOX2, MESP1, MESP2, MFACTOR, MGA, MGA::EVX1, MHOX(K2), MI, MIF1, MITF, MIXL1, MIZ1, MEX, MLXIPL, MMI, MNT, MNX1, MOP3, MR, MSANTD3, MSC, MSGN1, MSX1, MSX2, MTBZF, MTF1, MTF2, MTTF1, MXI1, MXIL, MYB, MYBL1, MYBL2, MYC, MYC1, MYCN, MYF3, MYF4, MYF5, MYF6, MYNN, MYOD, MYOD1, MYOG, MYRF, MZF1, N10(25, NANOG, NC2, NCI, NCX, NELF, NERI, NET, NEUROD1, NEUROD2, NEUROGI, NEUROG2, NF1A, NF1B, NF1X, NF4FA, NF4FB, NF4FC, NFA, NFAB, NFAT1, NFAT3, NFAT5, NFATC, NFATC1, NFATC2, NFATC3, NFATC4, NFATP, NFATX, NFCLEOA, NFCLEOB, NFDELTAE3A, NFDELTAE3B, NFDELTAE3C, NFDELTAE4A, NFDELTAE4B, NFDELTAE4C, NFE, NFE2, NFE2L1, NFE2L2, NFE2P45, NFE3, NFE6, NFETAA, NFGMA, NFGMB, NFI11A, NFIA, NFIB, NFIC, NFIC::TLX1, NFIL2A, NFIL2B, NFIL3, NFIX, NFJUN, NFKAPPAB, NFKAPPAB(LIKE), NFKAPPAB1, NFKAPPAB2, NFKAPPAB2(P49), NFKAPPAB2PRECURSOR, NFKAPPAE1, NFKAPPAE2, NFKAPPAE3, NFKB1, NFKB2, NFMHCIIA, NFMHCIIB, NFMUE1, NFMUE2, NFMUE3, NFNF1, NFS, NFX, NFX1, NFX2, NFX3, NFXC, NFYA, NFYB, NFYC, NFZC, NFZZ, NHLH1, NHLH2, NHP1, NHP2, NHP3, NHP4, NKX21, NKX22, NKX23, NKX24, NKX25, NKX28, NKX2B, NKX2C, NKX2G, NKX31, NKX32, NKX3A, NKX3AV1, NKX3AV2, NKX3AV3, NKX3AV4, NKX3B, NKX61, NKX62, NKX63, NKX6A, NMI, NMYC, NOBOX, NOCT2ALPHA, NOCT2BETA, NOCT3, NOCT4, NOCT5A, NOCTSB, NOTO, NPAS2, NPTCII, NR1D1, NR1D2, NR1H2::RXRA, NR1H3, NR1H4, NR1H4::RXRA, NR1I2, NR1I3, NR2C1, NR2C2, NR2E1, NR2E3, NR2F1, NR2F2, NR2F6, NR3C1, NR3C2, NR4A1, NR4A2, NR4A2::RXRA, NR5A1, NR5A2, NR6A1, NRF1, NRF2, NRF2BETA1, NRF2GAMMA1, NRL, NRSFFORM1, NRSFFORM2, NTF, OCAB, OCT1, OCT2, OCT2.1, OCT2B, OCT2C, OCT4A, OCT4B, OCT5, OCT6, OCTAFACTOR, OCTAMERBINDINGFACTOR, OCTB2, OCTB3, OLIGI, OLIG2, OLIG3, ONECUTI, ONECUT2, ONECUT3, OSRI, OSR2, OTX1, OTX2, OVOL1, OVOL2, OZF, P107, P130, P28MODULATOR, P300, P38ERG, P45, P49ERG, P53, P55, P55ERG, P65DELTA, P67, PATZ1, PAX1, PAX2, PAX3, PAX3A, PAX3B, PAX4, PAX5, PAX6, PAX6/PD5A, PAX7, PAX8, PAX8A, PAX8B, PAX8C, PAX8D, PAX8E, PAX8F, PAX9, PBX1, PBX1A, PBX1B, PBX2, PBX3, PBX3A, PBX3B, PBX4, PC2, PC4, PCS, PDX1, PEA3, PEBP2ALPHA, PEBP2BETA, PGR, PHF1, PHOX2A, PHOX2B, PIT1, PITX1, PITX2, PITX3, PKNOX1, PKNOX2, PLAGI, PLAGL2, PLZF, POB, PONTIN52, POU1F1, POU2F1, POU2F1::SOX2, POU2F2, POU2F3, POU3F1, POU3F2, POU3F3, POU3F4, POU4F1, POU4F2, POU4F3, POU5F1, POU5FIB, POU6F1, POU6F2, PPARA, PPARA::RXRA, PPARALPHA, PPARBETA, PPARD, PPARG, PPARG::RXRA, PPARGAMMA1, PPARGAMMA2, PPUR, PR, PRA, PRB, PRD1BF1, PRDIBFC, PRDM1, PRDM14, PRDM4, PRDM6, PRDM9, PRECURSOR, PROP1, PROX1, PRRX1, PRRX2, PSE1, PTEFB, PTF, PTF1A, PTFALPHA, PTFBETA, PTFDELTA, PTFGAMMA, PU.1, PUBOXBINDINGFACTOR, PUBOXBINDINGFACTOR(BJAB), PUF, PURFACTOR, R1, R2, RARA, RARA::RXRA, RARA::RXRG, RARALPHA1, RARB, RARBETA, RARBETA2, RARG, RARGAMMA, RARGAMMA1, RAX, RAX2, RBAK, RBP60, RBPJ, RBPJKAPPA, REL, RELA, RELB, REST, RFX, RFX1, RFX2, RFX3, RFX4, RFX5, RFX7, RFXS, RFY, RHOXF1, RORA, RORALPHA1, RORALPHA2, RORALPHA3, RORB, RORBETA, RORC, RORGAMMA, ROX, RPF1, RPGALPHA, RREB1, RSRFC4, RSRFC9, RUNX1, RUNX2, RUNX3, RVF, RXRA, RXRA::VDR, RXRALPHA, RXRB, RXRBETA, RXRG, SALL4, SAP1A, SAP1B, SATB1, SCRT1, SCRT2, SF1, SHOX2A, SHOX2B, SHOXA, SHOXB, SHP, SIIIP110, SIIIP15, SIIIP18, SIM′, SIX1, SIX2, SIX3, SIX4, SIX5, SIX6, SKOR1, SKOR2, SMAD1, SMAD2, SMAD3, SMAD4, SMAD5, SNAIl, SNAI2, SNAI3, SOHLH2, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP2, SP3, SP4, SP5, SP8, SP9, SPDEF, SPHFACTOR, SPI1, SPIB, SPIC, SPIN, SPZ1, SRCAP, SREBF1, SREBF2, SREBP1A, SREBP1B, SREBP1C, SREBP2, SREZBP, SRF, SRPLSTAF50, SRY, STAT1, STAT1::STAT2, STAT1ALPHA, STATIBETA, STAT2, STAT3, STAT4, STAT5A, STAT5B, STAT6, T, T3R, T3RALPHA1, T3RALPHA2, T3RBETA, TAF(I)110, TAF(I)48, TAF(I)63, TAF(II)100, TAF(II)125, TAF(II)135, TAF(II)170, TAF(II)18, TAF(II)20, TAF(II)250, TAF(II)250DELTA, TAF(II)28, TAF(II)30, TAF(II)31, TAF(II)55, TAF(II)70ALPHA, TAF(II)70BETA, TAF(II)70GAMMA, TAFI, TAFII, TAFL, TAL1, TAL1::TCF3, TALIBETA, TAL2, TARFACTOR, TBP, TBR1, TBX1, TBX15, TBX18, TBX19, TBX1A, TBX1B, TBX2, TBX20, TBX21, TBX3, TBX4, TBX5, TBX6, TBXS(LONGISOFORM), TBXS(SHORTISOFORM), TBXT, TCF, TCF1, TCF12, TCF1A, TCF1B, TCF1C, TCF1D, TCF1E, TCF1F, TCF1G, TCF21, TCF2ALPHA, TCF3, TCF4, TCF4(K), TCF4B, TCF4E, TCF7, TCF7L1, TCF7L2, TCFBETA1, TCFL5, TEAD1, TEAD2, TEAD3, TEAD4, TEF, TEF1, TEF2, TEL, TET1, TFAP2A, TFAP2B, TFAP2C, TFAP2E, TFAP4, TFAP4::ETV1, TFAP4::FLI1, TFCP2, TFCP2L1, TFDP1, TFE3, TFEB, TFEC, TFIIA, TFIIAALPHA/BETAPRECURSOR, TFIIAGAMMA, TFIIB, TFIID, TFIIE, TFIIEALPHA, TFIIEBETA, TFIIF, TFIIFALPHA, TFIIFBETA, TFIIH, TFIIH*, TFIIHCAK, TFIIHCYCLINH, TFIIHERCC2/CAK, TFIIHM015, TFIIHMAT1, TFIIHP34, TFIIHP44, TFIIHP62, TFIIHP80, TFIIHP90, TFIII, TFLFLTFLF2, TGIF, TGIF1, TGIF2, TGIF2LX, TGIF2LY, TGT3, THAP1, THAP11, THAP12, THRA, THRA1, THRB, TIF2, TIGD1, TLE1, TLX2, TLX3, TMF, TOPORS, TP53, TP63, TP73, TR2, TR211, TR29, TR3, TR4, TRAP, TREB1, TREB2, TREB3, TREF1, TREF2, TRF(2), TRPS1, TTF1, TWISTI, TXREBP, TXREF, UBF, UBP1, UEF1, UEF2, UEF3, UEF4, UNCX, USF1, USF2, USF2B, VAV, VAX2, VDR, VENTX, VEZF1, VHNF1A, VHNF1B, VHNF1C, VITF, VSX1, VSX2, WSTF, WT1, WT1DE12, WT1I, WT1IDE12, WT1IKTS, WT1KTS, X2BP, XBP1, XPA, XWV, XX, YAF2, YB1, YBX1, YEBP, YY1, YY2, ZBED1, ZBED2, ZBTB12, ZBTB14, ZBTB17, ZBTB18, ZBTB2, ZBTB20, ZBTB22, ZBTB26, ZBTB32, ZBTB33, ZBTB37, ZBTB42, ZBTB43, ZBTB44, ZBTB45, ZBTB48, ZBTB49, ZBTB6, ZBTB7A, ZBTB7B, ZBTB7C, ZEB, ZEB1, ZF1, ZF2, ZFHX2, ZFHX3, ZFP1, ZFP14, ZFP28, ZFP3, ZFP41, ZFP42, ZFP57, ZFP64, ZFP69, ZFP69B, ZFP82, ZFP90, ZFX, ZHX1, ZIC1, ZIC2, ZIC3, ZIC4, ZIC5, ZID, ZIK1, ZIM2, ZIM3, ZKSCAN1, ZKSCAN2, ZKSCAN3, ZKSCAN5, ZKSCAN7, ZNF10, ZNF100, ZNF101, ZNF114, ZNF12, ZNF121, ZNF124, ZNF132, ZNF133, ZNF134, ZNF135, ZNF136, ZNF140, ZNF141, ZNF143, ZNF146, ZNF148, ZNF154, ZNF157, ZNF16, ZNF17, ZNF174, ZNF175, ZNF177, ZNF18, ZNF180, ZNF181, ZNF182, ZNF184, ZNF189, ZNF19, ZNF197, ZNF2, ZNF200, ZNF202, ZNF205, ZNF211, ZNF212, ZNF213, ZNF214, ZNF22, ZNF222, ZNF223, ZNF224, ZNF225, ZNF23, ZNF232, ZNF235, ZNF24, ZNF248, ZNF25, ZNF250, ZNF254, ZNF257, ZNF26, ZNF260, ZNF263, ZNF264, ZNF266, ZNF267, ZNF273, ZNF274, ZNF276, ZNF28, ZNF280A, ZNF281, ZNF282, ZNF283, ZNF284, ZNF285, ZNF287, ZNF296, ZNF3, ZNF30, ZNF300, ZNF302, ZNF304, ZNF311, ZNF317, ZNF32, ZNF320, ZNF322, ZNF324, ZNF324B, ZNF329, ZNF331, ZNF333, ZNF334, ZNF335, ZNF337, ZNF33A, ZNF33B, ZNF34, ZNF341, ZNF343, ZNF345, ZNF35, ZNF350, ZNF354A, ZNF354B, ZNF37A, ZNF382, ZNF383, ZNF384, ZNF385D, ZNF394, ZNF396, ZNF398, ZNF41, ZNF410, ZNF415, ZNF416, ZNF417, ZNF418, ZNF419, ZNF423, ZNF425, ZNF429, ZNF430, ZNF431, ZNF432, ZNF433, ZNF436, ZNF439, ZNF44, ZNF440, ZNF441, ZNF442, ZNF443, ZNF444, ZNF445, ZNF449, ZNF45, ZNF454, ZNF460, ZNF467, ZNF468, ZNF479, ZNF480, ZNF483, ZNF484, ZNF485, ZNF486, ZNF487, ZNF490, ZNF492, ZNF496, ZNF501, ZNF502, ZNF506, ZNF513, ZNF519, ZNF524, ZNF525, ZNF527, ZNF528, ZNF529, ZNF530, ZNF534, ZNF540, ZNF543, ZNF547, ZNF548, ZNF549, ZNF550, ZNF552, ZNF554, ZNF555, ZNF558, ZNF561, ZNF562, ZNF563, ZNF564, ZNF565, ZNF566, ZNF567, ZNF570, ZNF571, ZNF573, ZNF574, ZNF580, ZNF582, ZNF584, ZNF585A, ZNF586, ZNF587, ZNF594, ZNF595, ZNF596, ZNF597, ZNF605, ZNF610, ZNF611, ZNF613, ZNF614, ZNF615, ZNF616, ZNF619, ZNF620, ZNF621, ZNF626, ZNF627, ZNF641, ZNF652, ZNF653, ZNF655, ZNF660, ZNF662, ZNF667, ZNF669, ZNF671, ZNF674, ZNF675, ZNF677, ZNF680, ZNF681, ZNF682, ZNF684, ZNF69, ZNF692, ZNF695, ZNF7, ZNF701, ZNF704, ZNF705G, ZNF707, ZNF708, ZNF71, ZNF711, ZNF713, ZNF714, ZNF716, ZNF730, ZNF736, ZNF737, ZNF74, ZNF740, ZNF749, ZNF75A, ZNF75D, ZNF76, ZNF764, ZNF765, ZNF766, ZNF768, ZNF77, ZNF770, ZNF771, ZNF774, ZNF776, ZNF777, ZNF778, ZNF780A, ZNF782, ZNF783, ZNF784, ZNF785, ZNF786, ZNF787, ZNF789, ZNF79, ZNF790, ZNF791, ZNF792, ZNF793, ZNF799, ZNF8, ZNF805, ZNF808, ZNF81, ZNF816, ZNF821, ZNF823, ZNF84, ZNF85, ZNF860, ZNF879, ZNF880, ZNF891, ZNF90, ZNF93, ZNF98, ZSCAN1, ZSCAN16, ZSCAN22, ZSCAN23, ZSCAN29, ZSCAN30, ZSCAN31, ZSCAN4, ZSCAN5, ZSCAN5C, ZSCAN9, and ZZZ3 and the like and others which may be identified in the literature.

One of skill in the art can readily identify transcription factors using databases and literature sources. In addition to the above databases, transcription factors are identified in US 2022/0214356 hereby incorporated by reference in its entirety for the description of transcription factors.

### Double Stranded DNA Deaminases

According to one aspect, exemplary double stranded DNA deaminases include DddA, also known in the art as BadTF1. See Mok, B. Y. et al., (2020). A bacterial cytidine deaminase toxin enables CRISPR-free mitochondrial base editing. Nature, 583(7817), 631-637 hereby incorporated by reference in its entirety for the teaching of DddA. DddA (BadTF1) belongs to SCP1.201-like deaminase subfamily.

According to one aspect, exemplary double stranded DNA deaminases include BadTF3. See Marcos H de Moraes et al., (2021) An interbacterial DNA deaminase toxin directly mutagenizes surviving target populations eLife 10:e62967. BadTF3 belongs to Pput_2613-like deaminase subfamily.

According to one aspect, exemplary double stranded DNA deaminases include DddA11. See Mok, B. Y., et al., (2022). CRISPR-free base editors with enhanced activity and expanded targeting scope in mitochondrial and nuclear DNA. Nature Biotechnology, 1-10, hereby incorporated by reference in its entirety for the teaching of DddA11.

It is known in the art that deaminases have been classified for identification. See Iyer L M, et al., Evolution of the deaminase fold and multiple origins of eukaryotic editing and mutagenic nucleic acid deaminases from bacterial toxin systems[J]. Nucleic acids research, 2011, 39(22): 9473-9497, hereby incorporated by reference in its entirety for classification and identification of deaminases. DddA of the present disclosure belongs to SCP1.201 clade. BadTF3 of the present disclosure belongs to Pput_2613-like clade. It is to be understood that the specific dsDNA deaminases described herein are exemplary only. The present disclosure contemplates mutants, variants, derivatives and modifications of dsDNA deaminases that exhibit enzymatic activity. The present disclosure contemplates the identification by those skilled in the art of other dsDNA deaminases, as well as mutants, variants, derivatives and modifications thereof that exhibit enzymatic activity.

In some examples, double stranded DNA deaminase can be derived from DddA deaminase, such as DddA6, DddA7 and other DddA variants. In other examples, the double stranded DNA deaminase can be derived from BadTF2 and other BadTF2 variants. In other examples, the double stranded DNA deaminase can be derived from BadTF3 and other BadTF3 variants. In further examples, the deaminase can be derived from bacterial toxins, such as Pput_2613 family deaminases, SCP1.201-like family deaminases, DYW-like family deaminases, BURPS668_1122-like family deaminases, YwgJ-like family deaminases, MafB19-like family deaminases, sce3516-like family deaminases, BH3703-like deaminases, WD0512-like family deaminases, and the like.

It is to be understood that aspects of the present disclosure include mutants, variants, truncations, modifications and derivatives of the full length dsDNA deaminases described herein and known to those of skill in the art, which exhibit deaminase activity. Methods of mutating, varying, truncating, modifying or derivatizing known dsDNA deaminases are known to those of skill in the art. Accordingly, the present disclosure contemplates that a dsDNA deaminase is one which exhibits deaminase activity with respect to a double stranded nucleic acid, regardless of whether it is naturally occurring or is a modified, mutated, varied, truncated, derivatized or evolved version of a naturally occurring dsDNA deaminase. Aspects of the present disclosure provide nucleic acid and amino acid sequences for various known dsDNA deaminases. Embodiments of the present disclosure include nucleic acid and amino acid sequences having 75% homology, 80% homology, 85% homology, 90% homology, 91% homology, 92% homology, 93% homology, 94% homology, 95% homology, 96% homology, 97% homology, 98% homology, 99% homology, 99.5% homology, 99.6% homology, 99.7% homology, 99.8% homology, or 99.9% homology to full length sequences of dsDNA deaminases disclosed herein. Based on the present disclosure, one of skill is able to identify dsDNA deaminases with percent homology to known dsDNA deaminases with deaminase activity on dsDNA or otherwise test such dsDNA deaminases with percent homology to known dsDNA deaminases with deaminase activity for deaminase activity.

### In Vitro Transposition

According to certain aspects, chromatin DNA treated with a dsDNA deaminase is processed using a transposition method which may be referred to in the art as transposome mediated fragmentation or “tagmentation”). In a tagmentation method, transposomes are prepared with DNA that is afterwards cut so that the transposition events result in fragmented DNA with adapters. In such methods, target DNA is simultaneously fragmented and tagged producing fragments tagged with desired DNA sequences for downstream processing. According to one aspect, a library is produced using an in vitro transposition system is utilized with the Nextera technology of Illumina, Inc, to simultaneously fragment DNA and tag each fragment with appropriate sequences for next-generation sequencing. See US20110287435 hereby incorporated by reference for disclosing tagmentation methods. For additional useful methods to make sequencing libraries, see Single-cell chromatin accessibility reveals principles of regulatory variation. Nature, 523(7561), 486-490) (2007); Massively multiplex single-cell Hi-C. Nature Methods, 14(3), 263-266) (2017); See also, useful transposition methods described in WO2016/073690 and WO2018217912 hereby incorporated by reference in their entireties.

According to certain aspects, an exemplary transposon system includes Tn5 transposase, Mu transposase, Tn7 transposase or IS5 transposase and the like. Other useful transposon systems are known to those of skill in the art and include Tn3 transposon system (see Maekawa, T., Yanagihara, K., and Ohtsubo, E. (1996), A cell-free system of Tn3 transposition and transposition immunity, Genes Cells 1, 1007-1016), Tn7 transposon system (see Craig, N. L. (1991), Tn7: a target site-specific transposon, Mol. Microbiol. 5, 2569-2573), Tn10 tranposon system (see Chalmers, R., Sewitz, S., Lipkow, K., and Crellin, P. (2000), Complete nucleotide sequence of Tn10, J. Bacteriol 182, 2970-2972), Piggybac transposon system (see Li, X., Burnight, E. R., Cooney, A. L., Malani, N., Brady, T., Sander, J. D., Staber, J., Wheelan, S. J., Joung, J. K., McCray, P. B., Jr., et al. (2013), PiggyBac transposase tools for genome engineering, Proc. Natl. Acad. Sci. USA 110, E2279-2287), Sleeping beauty transposon system (see Ivics, Z., Hackett, P. B., Plasterk, R. H., and Izsvak, Z. (1997), Molecular reconstruction of Sleeping Beauty, a Tcl-like transposon from fish, and its transposition in human cells, Cell 91, 501-510), Tol2 transposon system (seeKawakami, K. (2007), Tol2: a versatile gene transfer vector in vertebrates, Genome Biol. 8 Suppl. 1, S7.)

According to a general aspect, treated genomic DNA is contacted with Tn5 transposases each bound to a transposon DNA, to form a transposase/transposon DNA complex dimer called a transposome. The transposome bind to target locations along the treated genomic DNA and cleave the treated genomic DNA into a plurality of double stranded fragments with primer binding sites. Processing, such as extension and gap filling may take place to produce a double stranded product which is mixed with primers together with a DNA polymerase, nucleotides and amplification reagents, and the double stranded treated genomic DNA fragment is amplified. The amplicons are sequenced using, for example, high-throughput sequencing methods known to those of skill in the art.

### Nucleases

According to one aspect, a targeted chromatin region, such as an open chromatin region, may be enriched before or after treatment with a dsDNA deaminase using a nuclease. A nuclease is an enzyme capable of cleaving the phosphodiester bonds between nucleotides of nucleic acids. Nucleases variously effect single and double stranded breaks in their target molecules. There are two primary classifications based on the locus of activity. Exonucleases digest nucleic acids from the ends. Endonucleases act on regions in the middle of target molecules. According to certain aspects, a nuclease may be used to process DNA into fragments. Such processing may be before treatment of DNA with a dsDNA deaminase or after treatment of DNA with a dsDNA deaminase. Such fragments may then be amplified and/or sequenced as described herein. Exemplary nucleases include DNase (such as DNase I commercially available from Thermo Fisher), MNase (micrococcal nuclease commercially available from New England Biolabs) or a restriction endonuclease (such as FASTDIGEST commercially available from Thermo Fisher) and the like. According to one aspect, lysed cells can receive dsDNA deaminase treatment, and then be subjected to nuclease cleavage in order to enrich open chromatin regions for sequencing.

### Enriching for a Targeted Chromatin Region

According to one aspect, a targeted chromatin region, such as an open chromatin region, may be enriched before or after treatment with a dsDNA deaminase using methods known to those of skill in the art including ChIP-seq, ChIC, ChEC, ChEC-seq, CUT&TAG, CUT&RUN, Multi-CUT&Tag, NTT-seq, R loop CUT&Tag and the like, in addition to methods that use a transposase, such as Tn5 transposase.

ChIP-sequencing, also known as ChIP-seq, is a method used to analyze protein interactions with DNA. ChIP-seq combines chromatin immunoprecipitation (ChIP) with massively parallel DNA sequencing to identify the binding sites of DNA-associated proteins. It can be used to map global binding sites precisely for any protein of interest. Specific DNA sites in direct physical interaction with transcription factors and other proteins can be isolated by chromatin immunoprecipitation. ChIP produces a library of target DNA sites bound to a protein of interest. Massively parallel sequence analyses are used in conjunction with whole-genome sequence databases to analyze the interaction pattern of any protein with DNA, (See Johnson D S, et al., (June 2007). “Genome-wide mapping of in vivo protein-DNA interactions” (PDF). Science. 316 (5830): 1497-502) or the pattern of any epigenetic chromatin modifications. This can be applied to the set of ChIP-able transcription factors. See Whole-Genome Chromatin IP Sequencing (ChIP-Seq)” (PDF). Illumina, Inc. 26 Nov. 2007.

CUT&Tag-sequencing, also known as cleavage under targets and tagmentation, is a method used to analyze protein interactions with DNA. CUT&Tag-sequencing combines antibody-targeted controlled cleavage by a protein A-Tn5 fusion with massively parallel DNA sequencing to identify the binding sites of DNA-associated proteins. It can be used to map global DNA binding sites precisely for any protein of interest. See “CUT&Tag: a higher resolution, lower cost way to map chromatin”. Fred Hutchinson Cancer Research Center. 29 Apr. 2019.

CUT&RUN sequencing (see US 2022/0214356), also known as cleavage under targets and release using nuclease, is a method used to analyze protein interactions with DNA. CUT&RUN sequencing combines antibody-targeted controlled cleavage by micrococcal nuclease with massively parallel DNA sequencing to identify the binding sites of DNA-associated proteins. It can be used to map global DNA binding sites precisely for any protein of interest. See “Lay off the ChIPs: CUT&RUN instead”. Fred Hutchinson Cancer Research Center. 20 Feb. 2017.

ChIC detects the binding sites of transcription factors in the genome by targeting a modified micrococcal nuclease (MNase), conjugated with protein A (pA-MN), using a specific antibody. The modified MNase specifically cleaves DNA at regions interacting with a protein of interest only when Ca2+ ions are present, therefore allowing for controlled DNA cleavage at the antibody binding site. This approach allows for mapping proteins with a 100-200 bp resolution and excellent specificity. See Schmid M, Durussel T, Laemmli U K. 2004. ChIC and ChEC. Molecular Cell. 16(1):147-15.

Chromatin endogenous cleavage (ChEC) may be combined with high-throughput sequencing in a method referred to as ChEC-seq. ChEC-seq relies on fusion of a chromatin-associated protein of interest to micrococcal nuclease (MNase) to generate targeted DNA cleavage in the presence of calcium in living cells. ChEC-seq is not based on immunoprecipitation and so circumvents potential concerns with crosslinking, sonication, chromatin solubilization, and antibody quality while providing high resolution mapping with minimal background signal. See Grunberg et al., J. Vis. Exp. 2017; (124) e55836, p. 1-9.

Other methods of enriching a target chromatin region are known to those of skill in the art and readily identified by literature search.

According to one aspect, lysed cells received dsDNA deaminase treatment, and then are subjected to ChIP-seq, ChIC, ChEC, ChEC-seq, CUT&TAG, CUT&RUN, Multi-CUT&Tag, NTT-seq, R loop CUT&Tag processing in order to enrich chromatin regions targeted by a specific binding agent, such as an antibody. Alternatively, lysed cells can be firstly subjected to ChIP-seq, ChIC, ChEC, ChEC-seq, CUT&TAG, CUT&RUN, Multi-CUT&Tag, NTT-seq, R loop CUT&Tag processing to enrich targeted chromatin region without amplification and sequencing, and then dsDNA deaminase treatment can be was applied to map TF footprinting in targeted regions.

### Amplification

In certain aspects, amplification is achieved using PCR. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). The term “polymerase chain reaction” (“PCR”) of Mullis (U.S. Pat. Nos. 4,683,195, 4,683,202, and 4,965,188) refers to a method for increasing the concentration of a segment of a target sequence without cloning or purification. This process for amplifying the target sequence includes providing oligonucleotide primers with the desired target sequence and amplification reagents, followed by a precise sequence of thermal cycling in the presence of a polymerase (e.g., DNA polymerase). The primers are complementary to their respective strands (“primer binding sequences”) of the double stranded target sequence. In general, to effect amplification, the double stranded target sequence is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”) and the target sequence is said to be “PCR amplified.”

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

Any oligonucleotide or polynucleotide sequence can be amplified with the appropriate set of primer molecules. Methods and kits for performing PCR are well known in the art. All processes of producing replicate copies of a polynucleotide, such as PCR or gene cloning, are collectively referred to herein as replication.

The expression “amplification” or “amplifying” refers to a process by which extra or multiple copies of a particular polynucleotide are formed. Amplification includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reactions are commercially available. Primers useful to amplify sequences from a particular gene region are preferably complementary to, and hybridize specifically to sequences in the target region or in its flanking regions and can be prepared using methods known to those of skill in the art. Nucleic acid sequences generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The term “amplification reagents” may refer to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

Other amplification methods, as described in British Patent Application No. GB 2,202,328, and in PCT Patent Application No. PCT/US89/01025, each incorporated herein by reference, may be used in accordance with the present disclosure. Emulsion PCR may be used in accordance with the present disclosure. Other suitable amplification methods include “race and “one-sided PCR.”. (Frohman, In: PCR Protocols: A Guide To Methods And Applications, Academic Press, N.Y., 1990, each herein incorporated by reference). Methods based on ligation of two (or more) oligonucleotides in the presence of nucleic acid having the sequence of the resulting “di-oligonucleotide,” thereby amplifying the di-oligonucleotide, also may be used to amplify DNA in accordance with the present disclosure (Wu et al., Genomics 4:560-569, 1989, incorporated herein by reference).

RNA to be amplified may be obtained from a single cell or a small population of cells. Methods described herein allow RNA to be amplified from any species or organism in a reaction mixture, such as a single reaction mixture carried out in a single reaction vessel. In one aspect, methods described herein include sequence independent amplification of RNA from any source including but not limited to human, animal, plant, yeast, viral, eukaryotic and prokaryotic RNA.

As used herein, the term “primer” generally includes an oligonucleotide, either natural or synthetic, that is capable, upon forming a duplex with a polynucleotide template, of acting as a point of initiation of nucleic acid synthesis, such as a sequencing primer, and being extended from its 3′ end along the template so that an extended duplex is formed. Primers include extension primers, amplification primers or reverse transcription primers.

The sequence of nucleotides added during the extension process is determined by the sequence of the template polynucleotide. Usually primers are extended by a DNA polymerase or reverse transcriptase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate or quasi-degenerate in sequence. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In one aspect, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

Particularly exemplary amplification methods include rolling cycle amplification (RCA); Multiple displacement amplification (MDA); loop-mediated isothermal amplification (LAMP); strand displacement amplification (SDA, see U.S. Pat. No. 5,744,311); nucleic acid sequence-based amplification (NASBA see U.S. Pat. No. 6,025,134); quantitative realtime PCR; reverse transcriptase PCR (RT-PCR); real-time PCR (rt PCR); real-time reverse transcriptase PCR (rt RT-PCR); nested PCR; transcription-free isothermal amplification (see U.S. Pat. No. 6,033,881), repair chain reaction amplification (see WO 90/01069); ligase chain reaction amplification (see European patent publication EP-A-320308); gap filling ligase chain reaction amplification (see U.S. Pat. No. 5,427,930); coupled ligase detection and PCR (see U.S. Pat. No. 6,027,889).

### Sequencing

The amplicons are sequenced using, for example, high-throughput sequencing methods known to those of skill in the art. Determination of the sequence of a nucleic acid sequence of interest can be performed using a variety of sequencing methods known in the art including, but not limited to, sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLiD, Helicos, Polonator platforms, Ion Torrent semiconductor sequencing technology, single-molecule real-time (SMRT) sequencing from Pacific Biosciences, Nanopore-based sequencing from Oxford Nanopore Technologies, and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172). Exemplary sequencing platforms useful with the present disclosure and adaptable to the methods described herein are described in Reuter et al., High-Throughput Sequencing Technologies, Mol. Cell (2015); 58(4): 586-597 hereby incorporated by reference in its entirety.

The amplified DNA can be sequenced by any suitable method. In particular, the amplified DNA can be sequenced using a high-throughput screening method, such as Applied Biosystems' SOLiD sequencing technology, or Illumina's Genome Analyzer. In one aspect of the invention, the amplified DNA can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

“Shotgun sequencing” refers to a method used to sequence very large amount of DNA (such as the entire genome). In this method, the DNA to be sequenced is first shredded into smaller fragments which can be sequenced individually. The sequences of these fragments are then reassembled into their original order based on their overlapping sequences, thus yielding a complete sequence. “Shredding” of the DNA can be done using a number of difference techniques including restriction enzyme digestion or mechanical shearing. Overlapping sequences are typically aligned by a computer suitably programmed. Methods and programs for shotgun sequencing a DNA library are well known in the art.

Particularly exemplary sequencing methods include Sanger sequencing (AB 13730x1 genome analyzer), pyrosequencing on a solid support (454 sequencing, Roche), sequencing by-synthesis with reversible terminations (ILLUMINA Genome Analyzer), DNA nanoball sequencing (DNBSEQ, MGI), sequencing-by-ligation (ABI SOLID) or sequencing-by-synthesis with virtual terminators (HELI SCOPE®). Other next generation sequencing techniques for use with the disclosed methods include Massively parallel signature sequencing (MPSS), Polony sequencing, Ion Torrent semiconductor sequencing, DNA nanoball sequencing, Heliscope single molecule sequencing, Single molecule real time (SMRT) sequencing, Pacbio sequencing and Nanopore DNA sequencing.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures and accompanying claims.

### Example I

### Plasmid Construction

Expression constructs for DddA toxin domain (DddAtox), DddA11 and BadTF3 were obtained from Genescript through gene synthesis service.

To generate a pETDuet-1-based expression construct for DddAtox, the DddAtox encoding sequence: ATGGGCAGCAGCCATCACCATCATCACCACAGCCAGGATCCGGGTAGCTATGCGCT GGGTCCGTATCAGATCTCTGCTCCGCAGCTGCCGGCATATAACGGTCAGACTGTTGG TACTTTCTATTATGTTAACGATGCTGGCGGTTTAGAAAGCAAAGTTTTCAGCTCTGGT GGTCCGACCCCGTATCCGAACTATGCTAACGCTGGTCACGTTGAAGGTCAGTCTGCT CTGTTCATGCGTGATAACGGTATCTCTGAAGGTCTGGTTTTCCATAACAACCCGGAA GGTACCTGTGGTTTTTGTGTTAACATGACCGAAACCCTGCTGCCGGAAAACGCTAAA ATGACCGTTGTTCCGCCGGAAGGTGCGATTCCGGTTAAACGTGGTGCTACCGGTGAA ACCAAAGTTTTCACCGGTAACTCTAACTCTCCGAAATCTCCGACCAAAGGTGGTTGC TAA (SEQ ID NO:1) (of which the translated protein sequence is: MGSSHHHiHHHSQDPGSYALGPYQISAPQLPAYNGQTVGTFYYVNDAGGLESKVFSSGG PTPYPNYANAGHVEGQSALFMRDNGISEGLVFHNNPEGTCGFCVNMTETLLPENAKMT VVPPEGAIPVKRGATGETKVFTGNSNSPKSPTKGGC) (SEQ ID NO:2), is synthesized and cloned into the MCS-1 (NcoI and HindIII sites, retaining an N-terminal hexahistidine tag).

The immunity protein DddAI encoding sequence: ATGTATGCGGATGACTTTGACGGGGAAATTGAGATTGATGAAGTTGATAGCCTAGTT GAGTTTCTGAGCCGTCGTCCGGCGTTCGATGCGAACAACTTCGTTCTGACCTTCGAA GAAAGCGGCTTCCCGCAGCTGAACATCTTCGCGAAAAACGATATCGCGGTTGTTTAC TACATGGATATCGGCGAAAACTTCGTTAGCAAAGGCAACAGCGCGAGCGGCGGCAC CGAAAAATTCTACGAAAACAAACTGGGCGGCGAAGTTGATCTGAGCAAAGATTGCG TTGTTAGCAAAGAACAGATGATCGAAGCGGCGAAACAGTTCTTCGCGACCAAACAG CGTCCGGAACAGCTGACCTGGAGCGAACTGTAA (SEQ ID NO:3) (of which the translated protein sequence is: MYADDFDGEIEIDEVDSLVEFLSRRPAFDANNFVLTFEESGFPQLNIFAKNDIAVVYYMD IGENFVSKGNSASGGTEKFYENKLGGEVDLSKDCVVSKEQMIEAAKQFFATKQRPEQLT WSEL) (SEQ ID NO:4), is synthesized and cloned into the MCS-2 (NdeI and XhoI sites, removing an C-terminal S-tag).

To generate a pETDuet-1-based expression construct for DddA11, the DddA11 encoding sequence: ATGGGCAGCAGCCATCACCATCATCACCACAGCCAGGATCCGGGTAGCTATGCGCT GGGTCCGTATCAGATCTCTGCTCCGCAGCTGCCGGCATATAACGGTCAGACTGTTGG TACTTTCTATTATGTTAACGATGCTGGCGGTTTAGAAAGCAAAGTTTTCATCTCTGGT GGTCCGACCCCGTATCCGAACTATGTTAGCGCTGGTCACGTTGAAGGTCAGTCTGCT CTGTTCATGCGTGATAACGGTATCTCTGAAGGTCTGGTTTTCCATAACAACCCGAAA GGTACCTGTGGTTTTTGTGTTAACATGATCGAAACCCTGCTGCCGGAAAACGCTAAA ATGACCGTTGTTCCGCCGGAAGGTGCGATTCCGGTTAAACGTGGTGCTACCGGTGAA ACCAAAGTTTTCATCGGTAACTCTAACTCTCCGAAATCTCCGACCAAAGGTGGTTGC TAA (SEQ ID NO:5) (of which the translated protein sequence is: MGSSHHHIHHHSQDPGSYALGPYQISAPQLPAYNGQTVGTFYYVNDAGGLESKVFISGG PTPYPNYVSAGHVEGQSALFMRDNGISEGLVFHNNPKGTCGFCVNMIETLLPENAKMT VVPPEGAIPVKRGATGETKVFIGNSNSPKSPTKGGC) (SEQ ID NO:6), is synthesized and cloned into the MCS-1 (BamHI and HindIII sites, retaining an N-terminal hexahistidine tag), and the immunity protein DddAI encoding sequence: ATGTATGCGGATGACTTTGACGGGGAAATTGAGATTGATGAAGTTGATAGCCTAGTT GAGTTTCTGAGCCGTCGTCCGGCGTTCGATGCGAACAACTTCGTTCTGACCTTCGAA GAAAGCGGCTTCCCGCAGCTGAACATCTTCGCGAAAAACGATATCGCGGTTGTTTAC TACATGGATATCGGCGAAAACTTCGTTAGCAAAGGCAACAGCGCGAGCGGCGGCAC CGAAAAATTCTACGAAAACAAACTGGGCGGCGAAGTTGATCTGAGCAAAGATTGCG TTGTTAGCAAAGAACAGATGATCGAAGCGGCGAAACAGTTCTTCGCGACCAAACAG CGTCCGGAACAGCTGACCTGGAGCGAACTGTAA (SEQ ID NO:7) (of which the translated protein sequence is: MYADDFDGEIEIDEVDSLVEFLSRRPAFDANNFVLTFEESGFPQLNIFAKNDIAVVYYMD IGENFVSKGNSASGGTEKFYENKLGGEVDLSKDCVVSKEQMIEAAKQFFATKQRPEQLT WSEL) (SEQ ID NO:8), is synthesized and cloned into the MCS-2 (NdeI and XhoI sites, removing an C-terminal S-tag).

To generate a pETDuet-1-based expression construct for BadTF3, the BadTF3 encoding sequence: ATGGGCAGCAGCCATCACCATCATCACCACAGCCAGGATCCTGGTTGGAAATTTTCT AACGGTAAACGCCGTCCGCCGCACAAAGCAACGGTAACTGTGACCGATAAAAACGG TGTCGTTAAACACAAAAGCAACCTGGTTTCTGGCAACATGACTGAAGCCGAAAAGA AACTGGGCTTCCCGAACAACTCCCTGGCGACCCACACCGAAAACCGTGCTACCCGC CTGATCGATCTGAACCAAGGTGATACTATGCTGATCGAGGGCCAATACCGTCCGTGT CCACGTTGTAAAGGTGCAATGCGCGTGAAAGCGGAGGAATCCGGTGCGAAAGTGAT CTACACCTGGCCAGAAGATGGTGACCTGAAAAAACGTGAATGGGAAGGCACTCCGT GCGACAAAAAATAA (SEQ ID NO:9) (of which the translated protein sequence is: MGSSHHHHHHSQDPGWKFSNGKRRPPHKATVTVTDKNGVVKHKSNLVSGNMTEAEK KLGFPNNSLATHTENRATRLIDLNQGDTMLIEGQYRPCPRCKGAMRVKAEESGAKVIYT WPEDGDLKKREWEGTPCDKK) (SEQ ID NO: 10), is synthesized and cloned into the MCS-1 (NcoI and HindIII sites, retaining an N-terminal hexahistidine tag).

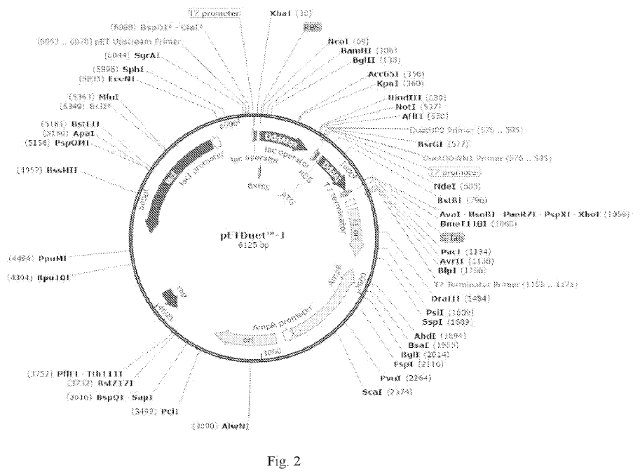

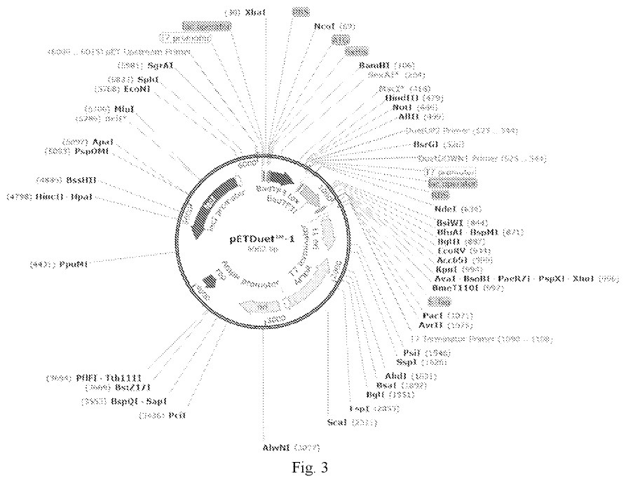

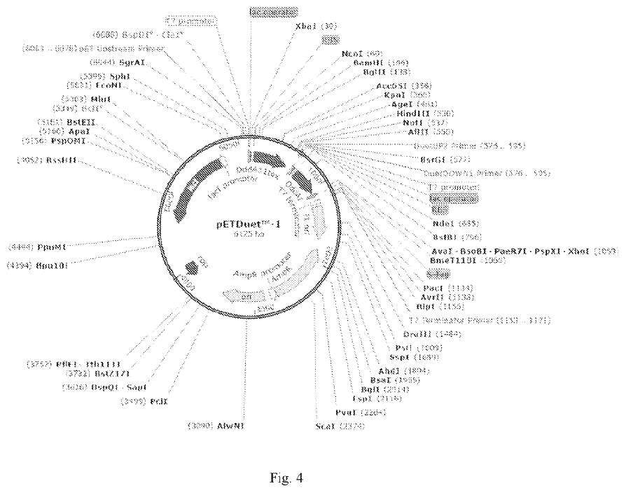

The immunity protein BadTF3I encoding sequence: ATGACCAAATCTAAAATGCTGAGCAACATCGTCATCCAGGAGGTCAAATTTGCGATC GAAGATTACTGCGCTATTCTGAGCTTCGCTTCTGACTCTTATGAAGTGCCGGAGCAG TATTTTATCATTACCCGTTCTACCACCGAACGTTCTGGCGGTATTCCGGAGGGCGAC ATCTACCTGGAATCTAACCTGTTTCTGGATTTTAACCCGTACGGCCTGAGCGGTTACC TGCTGTCTGAGCCGAACTGCGTAGATCTGCTGATCGAACCGAACAACTACGTTCGTC TGCGTCTGATCGAAAAAATCGATATCCTGGAAGTGGAAAACCACCTGAAATTTCTGT TCGACAACTAA (SEQ ID NO: 11) (of which the translated protein sequence is: MTKSKMLSNIVIQEVKFAIEDYCAILSFASDSYEVPEQYFIITRSTTERSGGIPEGDIYLESN LFLDFNPYGLSGYLLSEPNCVDLLIEPNNYVRLRLIEKIDILEVENHLKFLFDN) (SEQ ID NO:12), is synthesized and cloned into the MCS-2 (NdeI and KpnI sites, removing an C-terminal S-tag). The pETDuet-1::dddAtox+dddAI vector and pETDuet-1::badTF3tox+badTF3I were transformed into E. coli strains DH5α and BL21 and stored at −20° C. FIG. 2 depicts the vector map of pETDuet-1::dddAtox+dddAI. The inserted genes are driven by two independent lac operators. FIG. 3 depicts the vector map for pETDuet-1::badTF3tox+badTF3I. The inserted genes are driven by two independent lac operators. FIG. 4 depicts the vector map for pETDuet-1::dddA11+dddAI. The inserted genes are driven by two independent lac operators.

### Example II

### Bacterial Strains and Culture Conditions

Escherichia coli (E. coli) strains were grown in Lysogeny Broth (LB) at 37° C. or on LB medium solidified with agar (LBA, 1.5% w/v). The media was supplemented with the ampicillin (100 μg per ml) or IPTG (0.5 mM) if required. E. coli strains DH5α and BL21 were used for plasmid maintenance and protein expression, respectively.

### Example III

### Purification of DddAtox

The purification of DddAtox protein has been reported previously (Beverly et al. 2020, Nature, 583(7817): 631-637 doi: 10.1038/s41586-020-2477-4). Briefly, to purify the his-tagged DddAtox in complex with DddAI, E. coli BL21 (pETDuet-1::dddAtox+dddAI) was used to inoculate 2 L of LB broth in a 1:100 dilution and cultured overnight. After the culture grew to approximately OD600=0.6, 0.5 mM isopropyl β-D-1-thiogalactopyranoside (IPTG) was added and incubated for 16 hr at 18° C. with shaking. Bacterial cell pellets were collected through a centrifugation at 4000 g for 30 min, then resuspended in 50 ml of lysis buffer (50 mM Tris-HCl pH 8.0, 500 mM NaCl, 10 mM imidazole, 1 mg/mL lysozyme, and protease inhibitor cocktail). Bacterial cell pellets were then lysed by sonication (five pulses, 10 s each) and supernatant was separated from debris through a centrifugation at 25,000 g for 30 min. The his-tagged DddAtox-DddAI complex was purified from supernatant using a Nickel column. DddAtox-DddAI were eluted with 1 elution buffer (50 mM Tris-HCl pH 7.5, 300 mM imidazole, 500 mM NaCl, 30 mM imidazole, 1 mM DTT). The eluted DddAtox-DddAI complex was denatured by adding 50 ml 8 M urea denaturing buffer (50 mM Tris-HCl pH 7.5, 300 mM imidazole, 500 mM NaCl and 1 mM DTT) for 16 h at 4° C. The denatured proteins within 8 M urea denaturing buffer were loaded again on a Nickel column. The column was washed with 50 ml 8 M urea denaturing buffer to exclude any remaining DddAI. A sequential washing was applied to the column using 25 ml denaturing buffer with decreasing concentrations of urea (6 M, 4 M, 2 M, 1 M), and a last washing with wash buffer without urea. DddAtox that bound to the column was then eluted with 5 ml elution buffer. The eluted DddAtox was subjected to size-exclusion chromatography using fast protein liquid chromatography (FPLC) and gel filtration on a Superdex200 column (GE Healthcare) in sizing buffer (20 mM Tris-HCl pH 7.5, 200 mM NaCl, 1 mM DTT, 5% (w/v) glycerol). The purity of eluted DddAtox was evaluated by SDS-PAGE gel stained with Coomassie blue, then protein was stored at −80° C.

### Example IV

### Purification of DddAI 1

The purification of DddA11 protein was the same as DddAtoxin, as reported previously (Beverly et al. 2020, Nature, 583(7817): 631-637 doi: 10.1038/s41586-020-2477-4).

### Example V

### Purification of BadTF3

To purify the his-tagged BadTF3 in complex with BadTF3I, E. coli BL21 (pETDuet-1::BadTF3+BadTF3I) was used to inoculate 2 L of LB broth in a 1:100 dilution and cultured overnight. After the culture grew to approximately OD600=0.6, 0.5 mM IPTG was added and incubated for 16 hr at 18° C. with shaking. Bacterial cell pellets were collected through a centrifugation at 4000 g for 30 min, then resuspended in 50 ml of lysis buffer (50 mM Tris-HCl pH 8.0, 500 mM NaCl, 10 mM imidazole, 1 mg/ml lysozyme, and protease inhibitor cocktail). Bacterial cell pellets were then lysed by sonication (five pulses, 10 s each) and supernatant was separated from debris through a centrifugation at 25,000 g for 30 min. The his-tagged BadTF3-BadTF3I complex was purified from supernatant using a Nickel column. BadTF3-BadTF3I were eluted with 1 elution buffer (50 mM Tris-HCl pH 7.5, 300 mM imidazole, 500 mM NaCl, 30 mM imidazole, 1 mM DTT). The eluted BadTF3-BadTF3I complex was denatured by adding 50 ml 8 M urea denaturing buffer (50 mM Tris-HCl pH 7.5, 300 mM imidazole, 500 mM NaCl and 1 mM DTT) for 16 h at 4° C. The denatured proteins within 8 M urea denaturing buffer were loaded again on a Nickel column. The column was washed with 50 ml 8 M urea denaturing buffer to exclude any remaining BadTF3I. A sequential washing was applied to the column using 25 ml denaturing buffer with decreasing concentrations of urea (6 M, 4 M, 2 M, 1 M), and a last washing with wash buffer without urea. BadTF3 that bound to the column was then eluted with 5 ml elution buffer. The eluted BadTF3 was subjected to size-exclusion chromatography using fast protein liquid chromatography (FPLC) and gel filtration on a Superdex200 column (GE Healthcare) in sizing buffer (20 mM Tris-HCl pH 7.5, 200 mM NaCl, 1 mM DTT, 5% (w/v) glycerol). The purity of eluted BadTF3 was evaluated by SDS-PAGE gel stained with Coomassie blue, then protein was stored at −80° C.

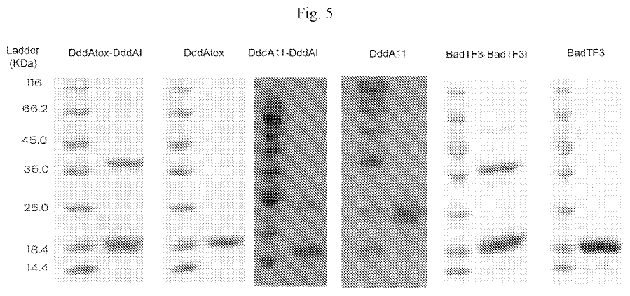

FIG. 5 depicts an SDS-PAGE gel stained with Coomassie blue of DddAtox-DddAI, DddAtox, DddA11-DddAI, DddA11, BadTF3-BadTF3I and BadTF3, respectively.

### Example VI

### DNA Deamination Activity Assays

Lambda DNA or genomic DNA extracted from drosophila S2 or K562 or GM12878 cell lines were used to evaluate the double-stranded DNA deamination activity. Reactions were performed in 10 μl of deamination buffer consisting of 20 mM Tris-HCl pH 7.4, 100 mM NaCl, 1 mM DTT, 50 ng DNA substrate and deaminase (20 μM, except as noted). Reactions were incubated for 1 hr or indicated time course at 37° C., followed by DNA purification using Zymo DNA Clean & Concentrator-5 kit. Purified DNA was subjected to library preparation using TruePrep DNA Library Prep Kit V2 for Illumina (Vazyme) as directed, expect replacing the polymerase mix with 1×Q5U PCR master mix plus Bst 3.0 polymerase (0.08 U per μl). The uracil conversion ratio at each cytosine site was calculated and averaged to evaluate the deamination activity in each possible sequence context.

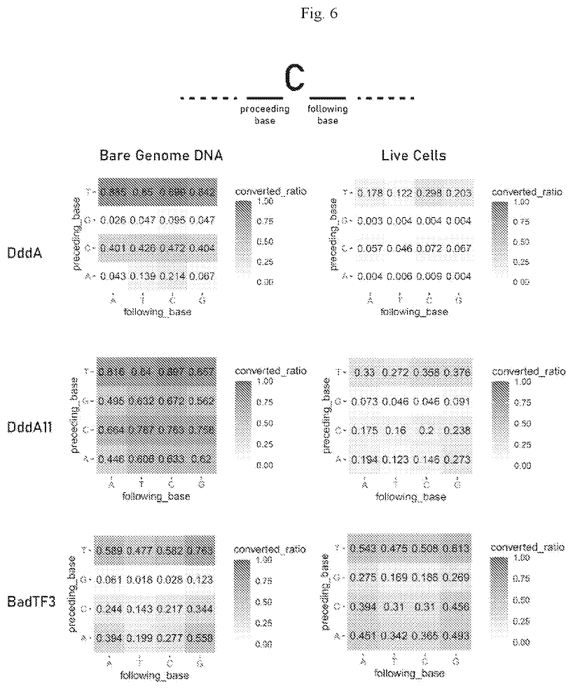

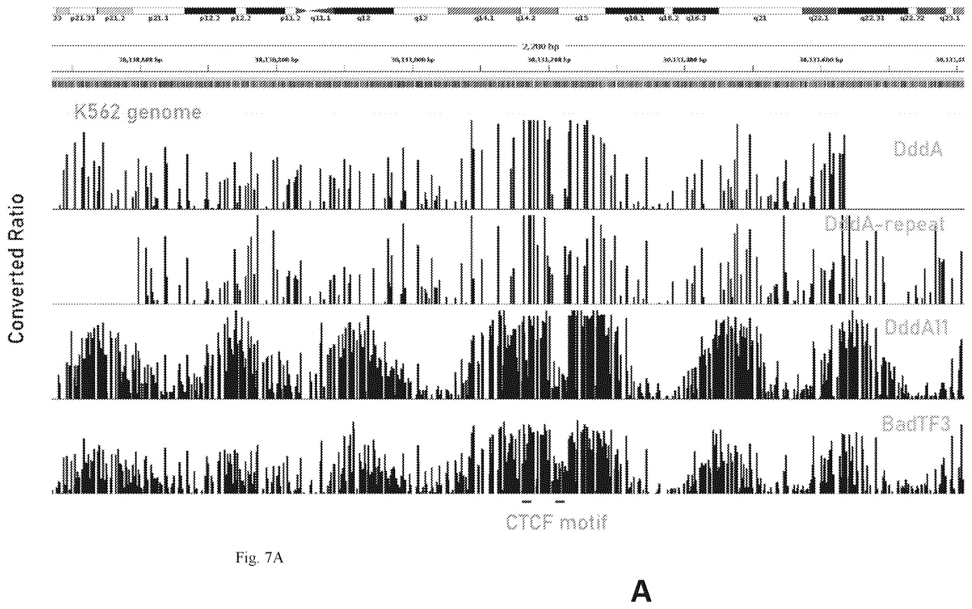

FIG. 6 depicts the conversion efficiency of cytosine to uracil in double stranded DNA treated by DddAtoxin, DddA11 protein or BadTF3 for one hour. Each square refers to a single cytosine with the four possible upstream nucleotide contexts and four possible downstream nucleotide contexts. The brightness of red color represents the conversion degree. The left side shows the average conversion efficiency of cytosine sites of bare genome DNA in vitro (drosophila genome for DddA and DddA11 and Lambda DNA for BadTF3). The right side shows the average conversion efficiency at cytosine sites of K562 genome DNA extracted from live cell experiments. The purified DddA specifically deaminated cytosine in TC or CC context (TCC converted to TUC, then U is regarded as T to initiate UC to UU conversion). Enzyme DddA11 and BadTF3 deaminated cytosine in TC, CC and AC and slightly GC context.

### Example VII

### Nuclei Preparation

To prepare nuclei, cells were spun down at 450 g for 5 min, followed by a washing using equal volume of cold 1×PBS and centrifugation at 500 g for 5 min at 4° C. A total of 30,000 cells were permeabilized using cold permeabilization buffer (10 mM Tris-HCl, pH 7.4, 10 mM NaCl, 3 mM MgCl2, 0.1% IGEPAL CA-630, 0.1% Tween-20, 0.1% digitonin). Immediately after permeabilization, nuclei were spun down at 550 g for 5 min at 4° C. Supernatant was carefully removed from the pellet after centrifugation.

### Example VIII

### Tagmentation Reaction

It is to be understood that tagmentation as described below can be carried out before DNA is treated with dsDNA deaminase or after DNA is treated with dsDNA deaminase. Prepare 2×TD buffer (20 mM TAPS ph8.5, 10 mM MgCl2, 20% DMF). Nuclei pellet was immediately resuspended in the transposase reaction mix (12.5 μL 2×TD buffer, 2 μL transposase (Vazyme, 1.25 μM) and 10 μL PBS with 0.1% digitonin). The transposition reaction was carried out for 30 min at 37° C. on a thermomixer at 800 rpm. After that, 100 μl ice-cold RSB was added to the mixer, which was followed by a centrifugation at 550 g for 5 min at 4° C. Supernatant was carefully removed from the pellet after centrifugation.

### Example IX

### DNA Deamination Reaction

Prepare 2×DRB buffer (20 mM Tris-HCl pH 7.5, 20 mM NaCl, 2 mM DTT). Nuclei pellet was immediately resuspended in the deaminase reaction mix. For DddAtox, the deaminase reaction mix contains 12 μl DRB and 18 μl DddA enzyme (50 uM, stored in sizing buffer). For DddA11, the deaminase reaction mix contains 8 μl DRB and 12 μl DddA11 enzyme. For BadTF3, the deaminase reaction mix contains 5 μl DRB and 5 μl BadTF3 enzyme (50 uM, stored in sizing buffer). The nuclei mix was incubated at 37° C. for 20 min. Directly following reaction the DNA was purified using a Zymo DNA Clean & Concentrator-5 kit.

### Example X

### Library Amplification

After DNA purification, library fragments were amplified using 1×Q5U PCR master mix, Bst 3.0 polymerase (0.08 U per μl) and 1.25 μM of Nextera PCR primers (forward and reverse), using the following PCR conditions: 65° C. for 5 min; 80° C. for 12 min; 98° C. for 2 min; and thermocycling at 98° C. for 15 s, 60° C. for 30 s and 72° C. for 1 min. It is recommended to monitor the PCR reaction using qPCR in order to stop amplification before saturation and avoid GC and size bias in PCR. After a pre-amplification of full libraries for five cycles, an aliquot of the PCR reaction was collected for qPCR. A total of 20 cycles qPCR was performed to determine the additional number of cycles needed for the remaining PCR reaction. Generally, a total of 10-12 cycles amplification yields high-quality libraries. The libraries were purified using a Zymo Select-a-Size DNA Clean & Concentrator Kit to collect DNA with fragment size above 200 bp. Size-selected libraries are ready for sequencing.

### Example XI

### Detection of Transcription Factor Footprints

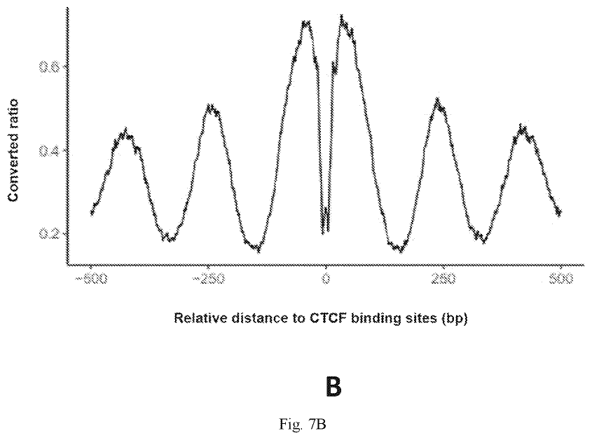

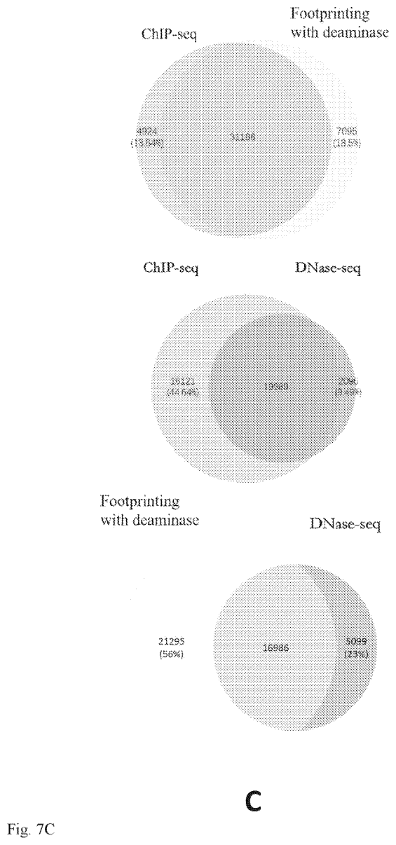

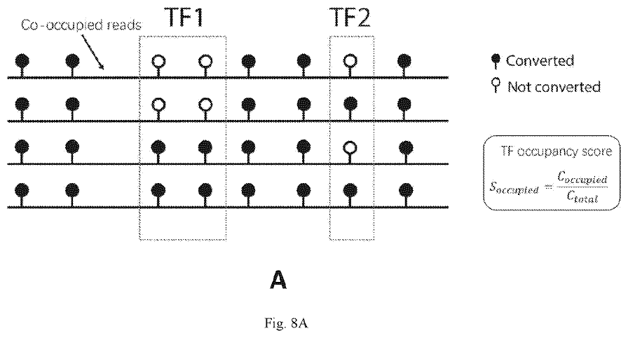

FIG. 7A depicts identification of transcription factor CTCF footprinting using the methods described herein in human K562 genome. Isolated nuclei were treated with DddA, DddA11, BadTF3 separately. The Y axis shows the conversion ratio of each cytosine site. The purple bar shows the CTCF binding motif FIG. 7B shows the average conversion ratio at merged CTCF binding motifs, a footprint is observed in the center. FIG. 7C depicts proportional Venn diagrams displaying the overlap between CTCF binding sites identified by the dsDNA deaminase method described herein, a ChIP-seq method and a DNase-seq method. A total of 31186 of CTCF binding sites detected by the dsDNA deaminase method (38281 in total) is accordant with the CTCF ChIP-seq method (36110 in total). In contrast, only 19989 out of 22085 binding sites identified by the DNase-seq method overlapped with binding sites identified by the ChIP-seq method. Those data demonstrate that the dsDNA deaminase method compares favorably with the ChIP-seq method and robustly identify a much larger number of TF binding sites.

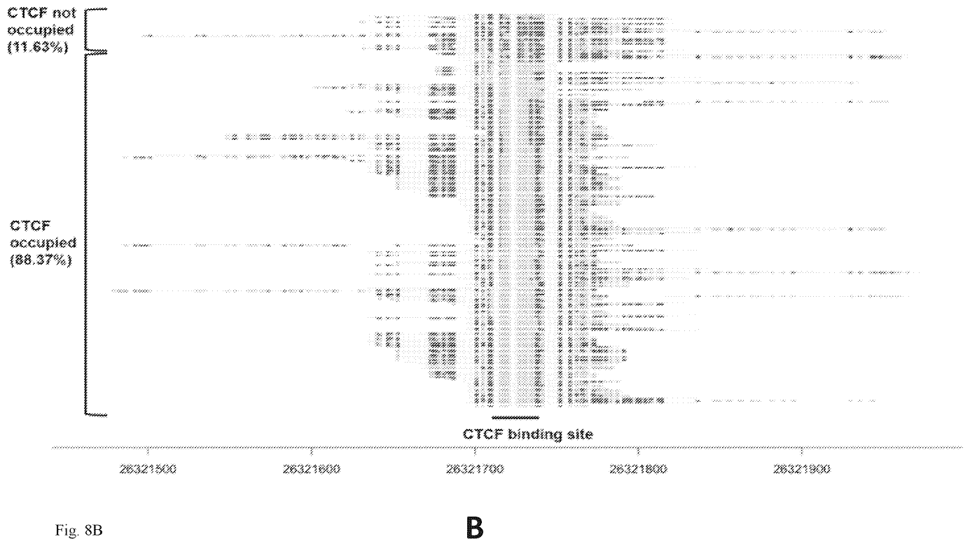

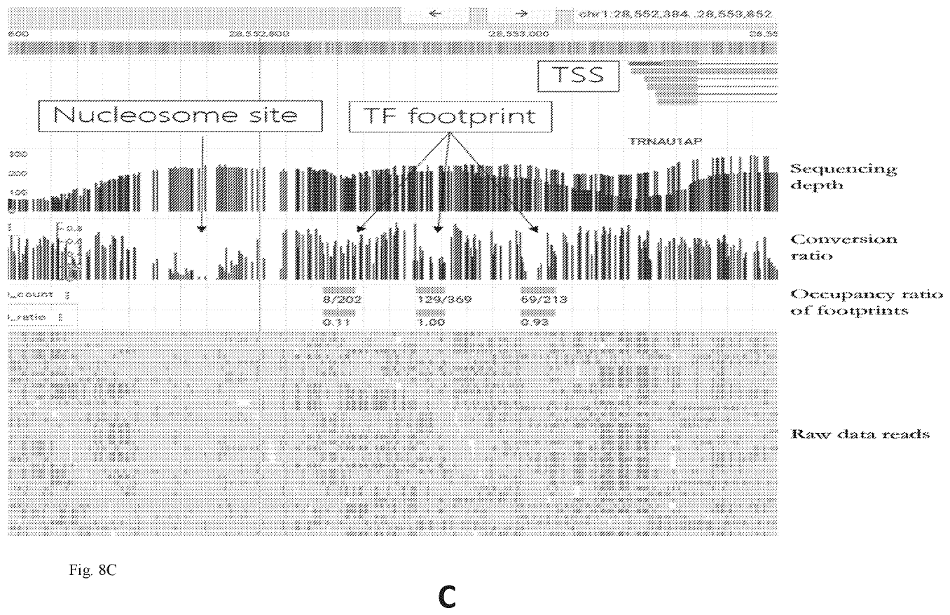

The methods described herein determine TF binding ratios by footprinting TFs within a single DNA molecule. FIG. 8A depicts in schematic the analysis of TF binding pattern at single molecule level using data obtained by methods described herein. For each sequencing read, the unconverted site is interpreted as TF binding region. Alternatively, the converted site is interpreted as the accessible region. Single read can be sorted according to the occupancy pattern over multiple genomic features, such as TFBS clusters. In FIG. 8B, each line represents a sequencing DNA read, and all the reads located in Chromosome 1:26321500-26321900 were cumulated. Each black dot represents a converted cytosine, and each gray dot represents a cytosine without conversion. If a DNA was occupied by a TF, for example CTCF in this case, the binding of the TF would prevent cytosine deamination at the specific binding site, whereas the upstream and downstream flanking cytosines would not be protected from deamination. On the other hand, if a DNA was not occupied by a TF, all cytosines would be accessible to dsDNA deaminase and subsequent deamination. Therefore, the TF binding at each DNA molecule was determined from which the TF binding ratio was calculated. The data shows that 88.37% DNA are occupied by CTCF at CTCF binding sites, whereas 11.63% are not. FIG. 8C depicts data demonstrating that the dsDNA deaminase method described herein is capable of simultaneous detecting of three TF binding sites and relative occupancy ratio in one promoter. Each dot within the raw reads refers to a cytosine conversion. In this case, the footprint closest to the transcription starting site has a highest binding ratio (only few conversions from cytosine to uracil occur in this region), whereas the most distal footprint has the lowest binding ratio among those three.

### Example XII

### Detection of Discrete Transcription Factor Footprints in a Single Cell

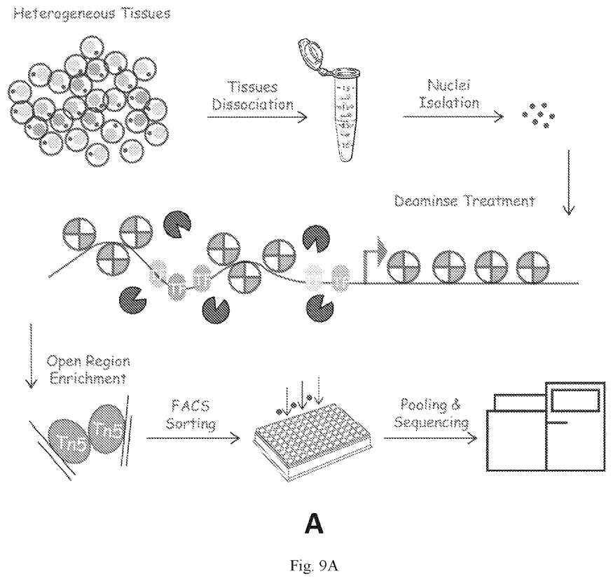

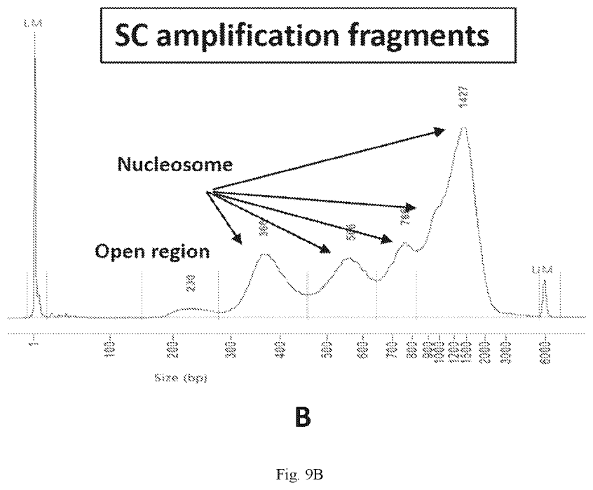

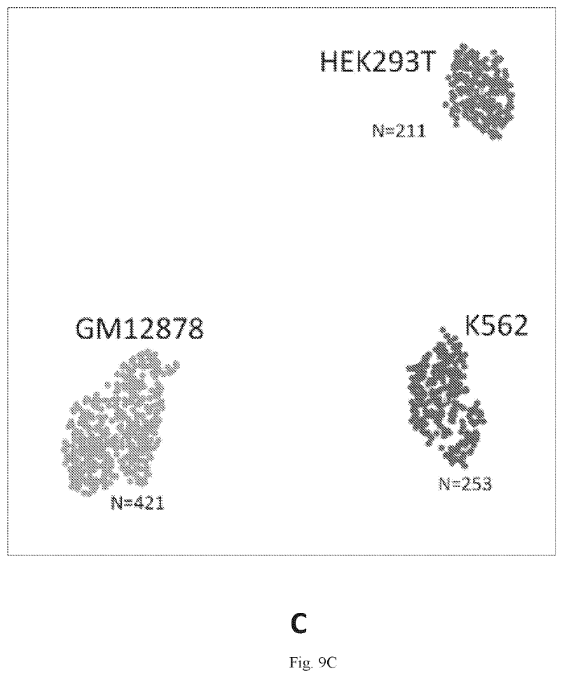

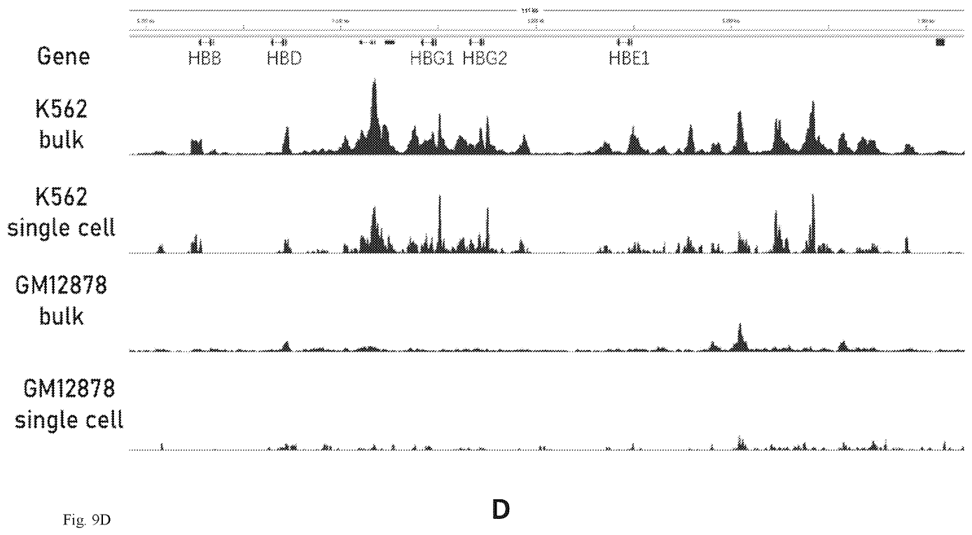

FIG. 9A depicts in schematic using methods described herein for detecting discrete TF footprints in chromatin DNA from a single cell. Heterogeneous tissues or samples were first dissociated into a single cell suspension. After cell lysis for nuclei isolation, deaminase enzyme DddA was added. Then with Tn5 transposition, the open region is added by universal adaptors and enriched. Single cell samples are obtained by FACS sorting. After gap filling with the help of BST, the library was amplified by Q5U and sequenced by Illumina sequencer. FIG. 9B shows the DNA fragment distribution of single cells after PCR amplification: the open region and nucleosome pattern could be identified clearly. FIG. 9C shows the cell typing results of single cell data from K562, GM12878, and Hek293T cell lines. The three cell types could be clustered well and the number of each cell type used was listed. FIG. 9D is a comparison of bulk and single cell data resulting from methods described herein as viewed by IGV software. The signal in single cell DATA correlates well with that in bulk ones, both in K562 and GM12878 cell line.

### Example XIII

### Sequences

The amino acid sequence of natural DddAtox is:

(SEQ ID NO: 15)
GSYALGPYQISAPQLPAYNGQTVGTFYYVNDAGGLESKVFSSGGPTPYP
NYANAGHVEGQSALFMRDNGISEGLVFHNNPEGTCGFCVNMTETLLPEN
AKMTVVPPEGAIPVKRGATGETKVFTGNSNSPKSPTKGGC

The amino acid sequence of purified DddAtox with N-terminal His tag is:

(SEQ ID NO: 2)
MGSSHHHHHHSQDPGSYALGPYQISAPQLPAYNGQTVGTFYYVNDAGGL
ESKVFSSGGPTPYPNYANAGHVEGQSALFMRDNGISEGLVFHNNPEGTC
GFCVNMTETLLPENAKMTVVPPEGAIPVKRGATGETKVFTGNSNSPKSP
TKGGC

The Genescript synthesized nucleotide sequence (optimized for bacterial expression) of DddAtox with N-terminal His tag is:

(SEQ ID NO: 1)
ATGGGCAGCAGCCATCACCATCATCACCACAGCCAGGATCCGGGTAGCT
ATGCGCTGGGTCCGTATCAGATCTCTGCTCCGCAGCTGCCGGCATATAA
CGGTCAGACTGTTGGTACTTTCTATTATGTTAACGATGCTGGCGGTTTA
GAAAGCAAAGTTTTCAGCTCTGGTGGTCCGACCCCGTATCCGAACTATG
CTAACGCTGGTCACGTTGAAGGTCAGTCTGCTCTGTTCATGCGTGATAA
CGGTATCTCTGAAGGTCTGGTTTTCCATAACAACCCGGAAGGTACCTGT
GGTTTTTGTGTTAACATGACCGAAACCCTGCTGCCGGAAAACGCTAAAA
TGACCGTTGTTCCGCCGGAAGGTGCGATTCCGGTTAAACGTGGTGCTAC
CGGTGAAACCAAAGTTTTCACCGGTAACTCTAACTCTCCGAAATCTCCG
ACCAAAGGTGGTTGCTAA

The amino acid sequence of natural DddAI (the same amino acid sequence for protein purification) is:

(SEQ ID NOs: 4, 8)
MYADDFDGEIEIDEVDSLVEFLSRRPAFDANNFVLTFEESGFPQLNIFA
KNDIAVVYYMDIGENFVSKGNSASGGTEKFYENKLGGEVDLSKDCVVSK
EQMIEAAKQFFATKQRPEQLTWSEL

The Genescript synthesized nucleotide sequence (optimized for bacterial expression) of DddAI is:

(SEQ ID NOs: 3, 7)
ATGTATGCGGATGACTTTGACGGGGAAATTGAGATTGATGAAGTTGATA
GCCTAGTTGAGTTTCTGAGCCGTCGTCCGGCGTTCGATGCGAACAACTT
CGTTCTGACCTTCGAAGAAAGCGGCTTCCCGCAGCTGAACATCTTCGCG
AAAAACGATATCGCGGTTGTTTACTACATGGATATCGGCGAAAACTTCG
TTAGCAAAGGCAACAGCGCGAGCGGCGGCACCGAAAAATTCTACGAAAA
CAAACTGGGCGGCGAAGTTGATCTGAGCAAAGATTGCGTTGTTAGCAAA
GAACAGATGATCGAAGCGGCGAAACAGTTCTTCGCGACCAAACAGCGTC
CGGAACAGCTGACCTGGAGCGAACTGTAA

The amino acid sequence of purified DddA11 with N-terminal His tag is:

(SEQ ID NOs: 6, 13)
MGSSHHHHHHSQDPGSYALGPYQISAPQLPAYNGQTVGTFYYVNDAGGL
ESKVFISGGPTPYPNYVSAGHVEGQSALFMRDNGISEGLVFHNNPKGTC
GFCVNMIETLLPENAKMTVVPPEGAIPVKRGATGETKVFIGNSNSPKSP
TKGGC

The Genescript synthesized nucleotide sequence (optimized for bacterial expression) of DddA11 is:

(SEQ ID NO: 5)
ATGGGCAGCAGCCATCACCATCATCACCACAGCCAGGATCCGGGTAGCT
ATGCGCTGGGTCCGTATCAGATCTCTGCTCCGCAGCTGCCGGCATATAA
CGGTCAGACTGTTGGTACTTTCTATTATGTTAACGATGCTGGCGGTTTA
GAAAGCAAAGTTTTCATCTCTGGTGGTCCGACCCCGTATCCGAACTATG
TTAGCGCTGGTCACGTTGAAGGTCAGTCTGCTCTGTTCATGCGTGATAA
CGGTATCTCTGAAGGTCTGGTTTTCCATAACAACCCGAAAGGTACCTGT
GGTTTTTGTGTTAACATGATCGAAACCCTGCTGCCGGAAAACGCTAAAA
TGACCGTTGTTCCGCCGGAAGGTGCGATTCCGGTTAAACGTGGTGCTAC
CGGTGAAACCAAAGTTTTCATCGGTAACTCTAACTCTCCGAAATCTCCG
ACCAAAGGTGGTTGCTAA

The Genescript synthesized protein sequence (optimized for bacterial expression) of is:

(SEQ ID NO: 6)
GSYALGPYQISAPQLPAYNGQTVGTFYYVNDAGGLESKVFISGGPTPYP
NYVSAGHVEGQSALFMRDNGISEGLVFHNNPKGTCGFCVNMIETLLPEN
AKMTVVPPEGAIPVKRGATGETKVFIGNSNSPKSPTKGGC

The natural amino acid sequence of BadTF3 is:

(SEQ ID NO: 14)
GWKFSNGKRRPPHKATVTVTDKNGVVKHKSNLVSGNMTEAEKKLGFPNN
SLATHTENRATRLIDLNQGDTMLIEGQYRPCPRCKGAMRVKAEESGAKV
IYTWPEDGDLKKREWEGTPCDKK

The amino acid sequence of purified BadTF3 with N-terminal His tag is:

(SEQ ID NO: 10)
MGSSHHHHHHSQDPGWKFSNGKRRPPHKATVTVTDKNGVVKHKSNLVSG
NMTEAEKKLGFPNNSLATHTENRATRLIDLNQGDTMLIEGQYRPCPRCK
GAMRVKAEESGAKVIYTWPEDGDLKKREWEGTPCDKK

The Genescript synthesized nucleotide sequence (optimized for bacterial expression) of BadTF3 is:

(SEQ ID NO: 9)
ATGGGCAGCAGCCATCACCATCATCACCACAGCCAGGATCCTGGTTGGA
AATTTTCTAACGGTAAACGCCGTCCGCCGCACAAAGCAACGGTAACTGT
GACCGATAAAAACGGTGTCGTTAAACACAAAAGCAACCTGGTTTCTGGC
AACATGACTGAAGCCGAAAAGAAACTGGGCTTCCCGAACAACTCCCTGG
CGACCCACACCGAAAACCGTGCTACCCGCCTGATCGATCTGAACCAAGG
TGATACTATGCTGATCGAGGGCCAATACCGTCCGTGTCCACGTTGTAAA
GGTGCAATGCGCGTGAAAGCGGAGGAATCCGGTGCGAAAGTGATCTACA
CCTGGCCAGAAGATGGTGACCTGAAAAAACGTGAATGGGAAGGCACTCC
GTGCGACAAAAAATAA

The amino acid sequence of natural BadTF3I (the same amino acid sequence for protein purification) is:

(SEQ ID NO: 12)
MTKSKMLSNIVIQEVKFAIEDYCAILSFASDSYEVPEQYFIITRSTTER
SGGIPEGDIYLESNLFLDFNPYGLSGYLLSEPNCVDLLIEPNNYVRLRL
IEKIDILEVENHLKFLFDN

The Genescript synthesized nucleotide sequence (optimized for bacterial expression) of BadTF3I is:

(SEQ ID NO: 11)
ATGACCAAATCTAAAATGCTGAGCAACATCGTCATCCAGGAGGTCAAAT
TTGCGATCGAAGATTACTGCGCTATTCTGAGCTTCGCTTCTGACTCTTA
TGAAGTGCCGGAGCAGTATTTTATCATTACCCGTTCTACCACCGAACGT
TCTGGCGGTATTCCGGAGGGCGACATCTACCTGGAATCTAACCTGTTTC
TGGATTTTAACCCGTACGGCCTGAGCGGTTACCTGCTGTCTGAGCCGAA
CTGCGTAGATCTGCTGATCGAACCGAACAACTACGTTCGTCTGCGTCTG
ATCGAAAAAATCGATATCCTGGAAGTGGAAAACCACCTGAAATTTCTGT
TCGACAACTAA.

### Example XIV

### Kits

The materials and reagents required for the disclosed method of determining TF footprints using a dsDNA deaminase may be assembled together in a kit. The kits of the present disclosure generally will include at least a dsDNA deaminase, transposase, nuclease, degradation enzyme, nucleotides, DNA polymerase, amplification primers and reagents, sequencing primers and reagents, and/or DNA enrichment reagents described herein which may be used to carry out the claimed method. In a preferred embodiment, the kit will also contain directions for treating chromatin DNA with dsDNA deaminase and processing and amplifying the treated chromatin DNA. In each case, the kits will preferably have distinct containers for each individual reagent, enzyme or reactant. Each agent will generally be suitably aliquoted in their respective containers. The container means of the kits will generally include at least one vial or test tube. Flasks, bottles, and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions are preferably provided with the kit.

### EMBODIMENTS

The present disclosure provides a method of determining a transcription factor binding site on genomic double stranded (ds) DNA of a cell, such as a eukaryotic cell, including contacting the genomic dsDNA with a dsDNA deaminase under conditions to convert cytosine of the genomic dsDNA to uracil, thereby creating treated genomic dsDNA, and identifying unconverted cytosine on the treated genomic dsDNA as a transcription factor binding site. According to one aspect, the genomic double stranded (ds) DNA is a gene. According to one aspect, the method includes identifying one or more unconverted cytosines as a transcription factor binding site. According to one aspect, the method includes identifying a plurality of unconverted cytosines as a transcription factor binding site. According to one aspect, the method includes identifying a plurality of unconverted cytosines as two or more transcription factor binding sites. According to one aspect, the genomic dsDNA includes a plurality of genes and the method further includes identifying a plurality of unconverted cytosines as a plurality of transcription factor binding sites. According to one aspect, the pattern of unconverted cytosines on the treated genomic DNA is correlated with DNA binding domains of transcription factors to identify one or more transcription factor binding sites. According to one aspect, the dsDNA deaminase is DddA, BadTF3 or DddA11 or a variant, mutant, derivative or modification thereof. According to one aspect, the dsDNA deaminase is an enzyme that is capable of converting cytosine to uracil on double stranded (ds) DNA. According to one aspect, treated genomic DNA is optionally amplified and sequenced to determine locations of unconverted cytosine and uracil. According to one aspect, treated genomic DNA is optionally amplified and sequenced to determine locations of unconverted cytosine and uracil which are compared to DNA binding patterns of transcription factors to identify transcription factor binding sites. According to one aspect, treated genomic DNA is optionally amplified and sequenced to determine locations of unconverted cytosine and uracil which are compared to DNA binding patterns of transcription factors to identify transcription factor binding sites and associated transcription factors. According to one aspect, the genomic dsDNA is a single DNA molecule from a single cell and the method further includes identifying a plurality of unconverted cytosines on the single DNA molecule as a plurality of transcription factor binding sites on the single DNA molecule. According to one aspect, the genomic double stranded (ds) DNA is processed into a plurality of DNA molecules which are analyzed or quantified for a transcription factor's relative binding ratio. According to one aspect, the treated genomic dsDNA is subjected to whole genome sequencing or targeted amplicon sequencing. According to one aspect, regions of converted cytosine to uracil flank a region of unconverted cytosine to identify a transcription factor footprint on an open region of the genomic dsDNA. According to one aspect, one or more open regions of the treated genomic dsDNA are enriched by tagmentation and amplification. According to one aspect, one or more open regions of the treated genomic dsDNA are enriched by nuclease digestion and amplification. According to one aspect, genomic dsDNA is obtained from a plurality of cells of the same cell type. According to one aspect, the treated genomic dsDNA is PCR amplified. According to one aspect, the treated genomic dsDNA is processed into fragments for sequencing. According to one aspect, the genomic dsDNA is treated with a dsDNA deaminase within a cell or within a nucleus isolated from a cell. According to one aspect, the genomic double stranded (ds) DNA is processed to enrich for open chromatin DNA. According to one aspect, the genomic double stranded (ds) DNA is processed to enrich for open chromatin DNA before treatment with the dsDNA deaminase. According to one aspect, the genomic double stranded (ds) DNA is treated with the dsDNA deaminase and then the treated genomic dsDNA is processed to enrich for open chromatin DNA.

### EQUIVALENTS

Other embodiments will be evident to those of skill in the art. It should be understood that the foregoing description is provided for clarity only and is merely exemplary. The spirit and scope of the present invention are not limited to the above examples, but are encompassed by the claims. All publications, patents and patent applications cited above are incorporated by reference herein in their entirety for all purposes to the same extent as if each individual publication or patent application were specifically indicated to be so incorporated by reference.

What is claimed is:

1. A method of determining a transcription factor binding site on genomic double stranded (ds) DNA of a eukaryotic cell comprising
contacting the genomic dsDNA with a dsDNA deaminase under conditions to convert cytosine of the genomic dsDNA to uracil, thereby creating treated genomic dsDNA, and
identifying unconverted cytosine on the treated genomic dsDNA as a transcription factor binding site.

2. The method of claim 1 wherein the genomic double stranded (ds) DNA is a gene.

3. The method of claim 1 comprising identifying one or more unconverted cytosines as a transcription factor binding site.

4. The method of claim 1 comprising identifying a plurality of unconverted cytosines as a transcription factor binding site.

5. The method of claim 1 comprising identifying a plurality of unconverted cytosines as two or more transcription factor binding sites.

6. The method of claim 1 wherein the genomic dsDNA comprises a plurality of genes and further comprising identifying a plurality of unconverted cytosines as a plurality of transcription factor binding sites.

7. The method of claim 1 wherein the pattern of unconverted cytosines on the treated genomic DNA is correlated with DNA binding domains of transcription factors to identify one or more transcription factor binding sites.

8. The method of claim 1 wherein the dsDNA deaminase is DddA, BadTF3 or DddA11 or a variant, mutant, derivative or modification thereof.

9. The method of claim 1 wherein the dsDNA deaminase is an enzyme that is capable of converting cytosine to uracil on double stranded (ds) DNA.

10. The method of claim 1 wherein treated genomic DNA is optionally amplified and sequenced to determine locations of unconverted cytosine and uracil.

11. The method of claim 1 wherein treated genomic DNA is optionally amplified and sequenced to determine locations of unconverted cytosine and uracil which are compared to DNA binding patterns of transcription factors to identify transcription factor binding sites.

12. The method of claim 1 wherein treated genomic DNA is optionally amplified and sequenced to determine locations of unconverted cytosine and uracil which are compared to DNA binding patterns of transcription factors to identify transcription factor binding sites and associated transcription factors.

13. The method of claim 1 wherein the genomic dsDNA is a single DNA molecule from a single cell and further comprising identifying a plurality of unconverted cytosines on the single DNA molecule as a plurality of transcription factor binding sites on the single DNA molecule.

14. The method of claim 1 wherein the genomic double stranded (ds) DNA is processed into a plurality of DNA molecules which are analyzed or quantified for a transcription factor's relative binding ratio.

15. The method of claim 1 wherein the treated genomic dsDNA is subjected to whole genome sequencing or targeted amplicon sequencing.

16. The method of claim 1 wherein regions of converted cytosine to uracil flank a region of unconverted cytosine to identify a transcription factor footprint on an open region of the genomic dsDNA.

17. The method of claim 1 wherein one or more open regions of the treated genomic dsDNA are enriched by tagmentation and amplification.

18. The method of claim 1 wherein one or more open regions of the treated genomic dsDNA are enriched by nuclease digestion and amplification.

19. The method of claim 1 wherein genomic dsDNA is obtained from a plurality of cells of the same cell type.

20. The method of claim 1 wherein the treated genomic dsDNA is PCR amplified.

21. The method of claim 1 wherein the treated genomic dsDNA is processed into fragments for sequencing.

22. The method of claim 1 wherein the genomic dsDNA is treated with a dsDNA deaminase within a cell or within a nucleus isolated from a cell.

23. The method of claim 1 wherein the genomic double stranded (ds) DNA is processed to enrich for open chromatin DNA.

24. The method of claim 1 wherein the genomic double stranded (ds) DNA is processed to enrich for open chromatin DNA before treatment with the dsDNA deaminase.

25. The method of claim 1 wherein the genomic double stranded (ds) DNA is treated with the dsDNA deaminase and then the treated genomic dsDNA is processed to enrich for open chromatin DNA.
